(19) United States
(12) Patent Application Publication
(10) Pub. No.: US 2024/0310657 A1
(43) Pub. Date: Sep. 19, 2024

(54) PERIPHERAL ANTI-DEFOCUS OPTICAL DEVICES

(71) Applicant: Hsiao-Ching TUNG, South Pasadena, CA (US)

(72) Inventor: Hsiao-Ching TUNG, South Pasadena, CA (US)

(21) Appl. No.: 18/273,444

(22) PCT Filed: Jan. 19, 2022
(86) PCT No.: PCT/US2022/013012

§ 371 Date: Jul. 20, 2023

Related U.S. Application Data
(60) Provisional application No. 63/139,746, filed on Jan. 20, 2021.

(51) Int. Cl. G02C 7/06; G02C 7/04

(52) U.S. Cl. CPC G02C 7/06; G02C 7/041; G02C 2202/24

(57) ABSTRACT

A contact or spectacle lens for correcting peripheral ocular defocus which has a center zone in a central portion for correcting refractive errors and an aspheric, annular anti-defocus zone adjacent to and extending radially outwardly from the center zone. The vertical meridian of the anti-defocus zone is less aspheric than the horizontal meridian of the anti-defocus zone, and the horizontal and vertical meridians are blended with progressively changing e-values to form a smooth optical surface.

### BACKGROUND

Many people experience difficulties with their vision due to a number of possible conditions. One of the most common sight problems is a condition known as myopia or nearsightedness. Myopia is a common condition where an eye cannot focus on far-away objects because the cornea of the eye is curved too steeply (i.e., the radius of curvature of the cornea is smaller than normal) or the axial length is too long to provide adequate focusing at the retina of the eye. Another condition is known as hyperopia or farsightedness. With hyperopia, the eye cannot focus on both far and near objects because the curvature of the cornea of the eye is too flat (i.e., the radius of curvature of the cornea is larger than normal) or the axial length is too short to provide adequate focusing at the retina of the eye. Another form of sight problem is astigmatism, in which unequal curvature of one or more refractive surfaces of the cornea prevents light rays from focusing clearly at one point on the retina, resulting in blurred vision. Presbyopia is the most common sight problem in adults 40 years and older due to aging and sclerosis of the crystalline lens. Presbyopia symptom may occur in younger age adults or children without crystalline lens sclerosis as part of visual efficiency problems, and is known as accommodative infacility or insufficiency.

Besides visual acuity problems, normal human vision also requires efficient eye teaming and visual efficiency skills for far and near tasks. Visual efficiency skills of the human eyes include three components, namely accommodation for focusing, vergence for binocular alignment, and oculomotor control for fixation and tracking of codes or objects. Intact visual acuity and visual efficiency skills are essential for effective information processing in the brain for the messages received by human eyes. There are also subdivision skills within each of the three visual efficiency skills. A well-trained eye doctor or ECP (eye care practitioner) can perform a comprehensive visual efficiency examination to discover and manage any defects. One of the well-known traditional examinations for visual efficiency is the OEP-21 points functional test. OEP is the abbreviation of the Optometric Extension Program Foundation located in California, USA. The evaluation of oculomotor control can be analyzed with tests including the NSUCO, SCCO, DEM™, and ReadAlyzer/Visagraph tests. There is increasing evidence of a relationship between visual efficiency and learning difficulties, such as ADHD/ADD (attention deficit disorder with/without hyperactivity) and dyslexia.

Approaches to correcting sight or visual acuity can include wearing spectacles, fitting contact lenses, Lasik surgery, and orthokeratology. The traditional approach to improving visual efficiency is usually through VT (visual training), which is operant conditioning training to teach the patient how to control their eyes for eye teaming. VT procedures can be supplemented with spherical lenses to relieve the focusing/accommodation stress, or with prism spectacles to supplement the vergence demand that comes with the excessive deviation angle. VT usually requires continuous office and home training for a couple of months or even one year to build up automaticity with the operant conditioning skills, which may regress and need frequent reinforcement to keep the skills up running. Supplemental spherical or prism devices may help initial VT in difficult cases, while eye teaming may adapt to the new focus or vergence demand the supplemental optical devices provided and worsen again with short symptom relief. Some visual efficiency problems, such as convergence insufficiency (CI), convergence excess (CE) and accommodation insufficiency (AI) may cause eyestrain and fasten myopia progression. Improving the visual efficiency may significantly slow down or stop myopia progression.

Notwithstanding the foregoing, there remains a need for a device or procedure that can better improve visual efficiency.

### SUMMARY

In one embodiment, the present invention provides improved visual efficiency through the use of an anti-defocus lens, such as a spectacle lens or a contact lens, for correcting peripheral ocular defocus. The lens has a center zone 20 in a central portion of the lens which has a lens power for correcting refractive errors, and an aspheric, annular anti-defocus (ADF) zone 21 adjacent to and extending radially outwardly from the center zone 20. The front surface or the back surface of the lens has a horizontal meridian and a vertical meridian, each of which has an e-value, and in the ADF zone the vertical meridian of the ADF zone is less aspheric than the horizontal meridian of the ADF zone. The vertical meridian of the ADF zone 21 can have a zero e-value, for example, and/or the e-value of the vertical meridian can be less than ½ of the e-value of the horizontal meridian. The curvature of the surface of the lens between the horizontal meridian and the vertical meridian is blended with progressively changing e-values to form a smooth optical surface. In one embodiment, the vertical meridian of the ADF zone 21 can be a single-vision curve having the same power as the central zone 20.

When the lens is a spectacle lens, the horizontal meridian and the vertical meridian can be on the front surface or back surface of the lens, preferably on the back surface. The center zone preferably has a diameter of between 1.5 and 4.0 mm, and the center zone and ADF zone together preferably have a diameter of between 18 and 28 mm. Such a spectacle lens can have an ADF zone with a horizontal meridian which is aspheric and progressively plus in power radially outward from an inner boundary of the ADF zone, and can have an anti-defocus power (ADP) of +1.00 to +20.0 diopters, with ADP being defined as the power difference between an outer periphery of the ADF zone 21 and an outer periphery of the center zone 20. Such a lens can be useful in the treatment of hyperopic ocular defocus. A spectacle lens useful in the treatment of myopic ocular defocus can be provided with an ADF zone 21 having a horizontal meridian which is aspheric and progressively minus in power radially outward from an inner boundary of the ADF zone, and which has an anti-defocus power (ADP) of −1.00 to −20.0 diopters.

When the lens is a contact lens, the center zone preferably has a diameter of between 0.5 and 1.0 mm, the ADF zone 21 preferably extends radially outwardly from the center zone for at least 3 to 4 mm, and the center zone 20 and annular ADF zone 21 together preferably have a diameter of between 6 and 10 mm. The lens can be a rigid corneal lens, a rigid scleral lens or a soft contact lens, for example. Such contact lenses can also further comprise an intermediate zone 24 coupled to and extending radially outwardly from the ADF zone 21, preferably having a zone width of 2.0-5.0 mm; a connecting zone 26 coupled to and extending radially outwardly from the intermediate zone 24 for bearing the contact lens on a cornea; and a peripheral zone 28 coupled to an outer periphery of the contact lens. In contact lens embodiments used in performing orthokeratology, the horizontal meridian and the vertical meridian are on the back surface of the lens in order to achieve corneal molding.

The e-values of the front surface or the back surface of the center zone 20 and the ADF zone 21 of contact lens embodiments can be merged to form an aspheric center-ADF zone 20-21, and the horizontal meridian and the vertical meridian of such a center-ADF zone 20-21 can be merged with a rotationally progressive e-value to form a center-ADF zone with a continuous smooth aspheric surface. In this embodiment, the vertical meridian can have an e-value of zero and can have a single-vision power throughout the center-ADF zone 20-21. The e-value of the horizontal meridian in this embodiment is preferably not zero, and can be between +0.1 e and +3.0 e. The rotationally progressive e-value Eₓ, along an axis X^{o} of the center-ADF zone, can be derived with the following formula:

$${E}_{x} = SIGN ( {XR}_{p} - {R}_{c} ) * {( ABS ( {XR}_{p}^{2} - {R}_{c}^{2} ) )}^{1 / 2} / d , \qquad ( Equation 2.2 )$$

where XRₚ is the radius of curvature at a point radially outward along axis X^{o} for a distance d, with XRₚ being derived from the following formula:

$${XR}_{p} = {HR}_{p} + {sin ( {X}^{o} )}^{2} * ( {VR}_{p} - {HR}_{p} ) , \qquad ( Equation 2.1 )$$

where R_{c} is the radius of curvature at the center of the contact lens and HRₚ is the radius of curvature at a point radially outward along the horizontal meridian for a distance d, with VRₚ being the radius of curvature at a point radially outward along the vertical meridian for a distance “1d”.

In contact lens embodiments used in the treatment of hyperopic ocular defocus, the horizontal meridian is progressively plus in power radially outward from a central portion of the lens and has an anti-defocus power (ADP) of +1.00 to +30.0 diopters. The front surface of the horizontal meridian of such a lens preferably has an e-value of between −0.1 e and −3.0 e, or the rear surface of the horizontal meridian preferably has an e-value of between +0.1 e and +3.0 e.

In contact lens embodiments used the treatment of myopic ocular defocus, the horizontal meridian is progressively minus in power radially outward from a central portion of the lens and has an anti-defocus power (ADP) of −1.00 to −30.0 diopters. The front surface of the horizontal meridian of such a lens preferably has an e-value of between +0.1 e and +3.0 e, or the rear surface of the horizontal meridian preferably has an e-value of between −0.1 e and −3.0 e.

The present invention also provides a method for improving or remediating visual efficiency problems with a lens as described above which corrects the peripheral ocular defocus in a subject's eye or eyes. The visual efficiency problem can be, for example, oculomotor dysfunction, accommodative dysfunction, vergence dysfunction, or abnormal sensory adaptation. This method can include determining an anti-defocus power (ADP) for a lens which has a center zone 20 in a central portion of the lens and an anti-defocus zone 21 adjacent to and extending radially outwardly from the center zone 20. The center zone has a central focal point to form a central image at the fovea retina for correcting refractive error, and the determined ADP is sufficient to offset peripheral ocular defocus and to realign peripheral images in the subject's eye in order to improve peripheral fusion and visual efficiency. Determining the anti-defocus power (ADP) in this method can further comprises checking baseline visual efficiency data for a subject; selecting ADF testing lenses based on the type of visual efficiency problem experienced by the subject; and testing raw ocular defocus strength by introducing the selected ADF testing lenses gradually from lower to higher ADP until an optimum ADP is determined which achieves maximum normalization of the visual efficiency data. A pair of spectacles or contact lenses having the optimum ADP can then be provided to the subject. The steps for determining an optimum ADP can be repeated after the subject has worn the provided spectacles or contact lenses for a predetermined period of time. For remediation of binocular efficiency problems or for myopia control in cases having hyperopic ocular defocus, the horizontal meridian of the ADF zone 21 is aspheric and progressively plus in power radially outward from an inner boundary of the ADF zone, which preferably has an ADP of +1.00 to +20.0 diopters. For treatment of myopic ocular defocus, the horizontal meridian of the ADF zone 21 is aspheric and progressively minus in power radially outward from an inner boundary of the ADF zone, which preferably has an ADP of −1.00 to −20.0 diopters. In one embodiment, a substantial anti-defocus effect is induced in a subject's eye for a minimum of 0.50 diopters relatively forward (more myopic) or backward (more hyperopic) in peripheral foci, measured at 10 degrees to each side (N10 and T10) of the subject's fovea retina. In another embodiment, a substantial anti-defocus effect is induced in a subject's eye for a minimum 2.00 diopters relatively forward (more myopic) or backward (more hyperopic) in peripheral foci, measured at 20 degrees to each side (N20 and T20) of the subject's fovea retina.

### FIGURES

FIG. 1 is a schematic illustration of the formation of an on-axis image on the eye.

FIG. 2 is a schematic illustration of the formation of a para-axis image on the eye.

FIG. 3 is a sectional view along line 3-3 of FIG. 4 of an anti-defocus (ADF) contact lens embodiment of the present device.

FIG. 4 is a plan view of the upper side (front surface) of the contact lens of FIG. 3.

FIG. 5 is a plan view of the lower side (back surface) of the contact lens of FIG. 3.

FIG. 6 is a sectional view along line 6-6 of FIG. 7 of a myopic anti-defocus (M-ADF) orthokeratology contact lens embodiment of the present device.

FIG. 7 is a planar view of the contact lens of FIG. 6.

FIG. 8 is a sectional view along line 8-8 of FIG. 9 of a hyperopic anti-defocus (H-ADF) orthokeratology contact lens embodiment of the present device.

FIG. 9 is a planar view of the contact lens of FIG. 8.

FIG. 10 is a front elevation view of a spectacle lens embodiment of the present device.

FIG. 11 is an illustration of Panum's fusion area.

The reference numbers in the figures identify the following features:

| Component | Reference Number |
|---|---|
| Contact lens | 10 |
| Spectacle lens | 11 |
| Cornea | 12 |
| Eye | 14 |
| Optical zone | 20 |
| Optical zone front | 20a |
| surface | |
| Optical zone back | 20b |
| surface | |
| Optical zone outer | 20c |
| periphery | |
| ADF zone | 21 |
| ADF zone front | 21a |
| surface | |
| ADF zone back | 21b |
| surface | |
| ADF zone outer | 21c |
| periphery | |
| ADF zone inner | 22 |
| boundary | |
| Intermediate zone | 24 |
| Connecting zone | 26 |
| Peripheral zone | 28 |
| Optical zone curve | 30 |
| Optical zone curve | 30a |
| (front surface) | |
| Optical zone base | 30b |
| curve (back | |
| surface) | |
| ADF zone curve | 31 |
| ADF zone curve | 31a |
| (front surface) | |
| ADF zone base | 31b |
| curve (back | |
| surface) | |
| Meridian | 50 |
| Horizontal | 51 |
| meridian | |
| Vertical meridian | 52 |
| Horopter | 60 |
| Horizontal | 62 |
| horopter | |
| Point on the | 63 |
| horizontal | |
| horopter | |
| Panum's fusion | 64 |
| area | |

### DESCRIPTION

### Definitions

As used herein, the following terms and variations thereof have the meanings given below, unless a different meaning is clearly intended by the context in which such term is used.

“AC/A ratio” means the ratio of the accommodative convergence AC (in prism diopters) to the stimulus to accommodation A (in diopters). The most common method of determining this ratio is by the gradient method (or gradient test) in which the phoria at near is measured after changing the accommodation with a spherical lens (usually +1.00 D or −1.00 D) placed in front of the two eyes. It is expressed as: AC/A=(α−α′)/F, where a is the phoria at near, and α′ is the phoria at the same distance but through a lens of power F. The deviation is measured in prism diopters, with + for esodeviation and − for exodeviation.

“Accommodative dysfunction” is a problem in the accommodative system, for the eye to change optical power, maintain a clear image, or focus on an object as its distance varies. There are several types of accommodative dysfunctions involving one or more of the following diagnoses: (1) accommodative insufficiency, (2) accommodative infacility, and (3) accommodative excess.

“Additional power” (ADD) is the refractive power difference between the far and near refractive powers of lenses. For spectacles, ADD is measured at a plane that is 12 mm in front of the anterior corneal surface. For any other device having a locus closer to or farther from the anterior cornea surface, the ADD is increased or reduced respectively for the distance with the vertex correction formula Fc=F/(1−xF), where Fc is the power corrected for vertex distance, F is the original lens power, and x is the change in vertex distance in meters.

“Anti-defocus (ADF) device” refers to an optical device, such as a pair of spectacles, soft contact lenses, rigid contact lenses, orthokeratology lenses or intraocular lenses, having a center optical zone 20 for correcting refractive errors of an eye and forming a central focal point at the fovea retina, and a peripheral anti-defocus zone 21 adjacent to and outward from the center optical zone 20, for eliciting backward (longer) or forward (shorter) peripheral foci to correct peripheral ocular defocus. A myopic anti-defocus (M-ADF) device is set for eliciting backward (longer) peripheral foci to offset myopic ocular defocus, and a hyperopic anti-defocus (H-ADF) device is set for eliciting forward (shorter) peripheral foci to offset hyperopic ocular defocus.

“Anti-defocus power” (ADP) of an ADF device is the power difference between (A) the outermost portion (periphery) 21c of the ADF zone 21 and (B) the outermost portion (periphery) 20c of the center zone 20 (equivalent to the inner boundary 22 of the ADF zone 21), i.e., ADP=A−B. ADP is plus in power for a H-ADF device and minus in power for a M-ADF device.

The “ADP effect” is the substantial anti-defocus effect when wearing an ADF device on a human eye, which can be measured with an open field refractometer or Shack-Hartmann system to compare the peripheral refraction with the ADF device relative to the baseline peripheral refraction without the ADF device. The ADP effect=A−B at a certain deviation angle from the central fovea. The deviation angles for measuring ADP are usually set horizontal 10°, 20°, 25°, 30° (degree) to each (nasal and temporal) side of the fovea retina.

“Associated phoria” is defined as the amount of prism required to reduce fixation disparity to zero.

The “back surface” of a lens refers to the surface through which light exits the lens in its normal and intended usage. For example, for a contact lens the back surface is the surface in contact with a subject's eye when worn by the subject.

“Base curve” refers to the curvature of the back surface of a contact lens.

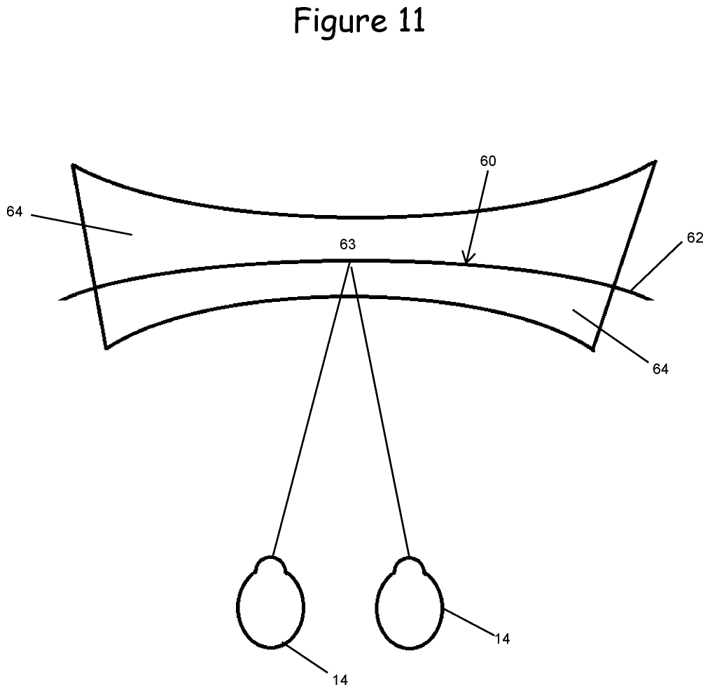

“Binocular fusion” is a type of vision allowing two eyes to be capable of perceiving a single binocular image of an individual's surroundings. Binocular fusion occurs only in a small portion of visual space around where the eyes are fixating. Running through the fixation point in the horizontal plane is a curved line called the empirical horizontal horopter 62. There is also an empirical vertical horopter tilted away from the eyes above the fixation point and towards the eyes below the fixation point. The horizontal and vertical horopters 60 mark the center of the volume of singleness of vision. Within this thin, curved volume, objects nearer and farther than the horopters are seen as single. The volume is known as Panum's fusion area 64 (see FIG. 11). Outside of Panum's fusion area, double vision occurs.

“Convergence” is a phenomenon in which the eyes rotate inwardly to focus on an object, and the degree to which they rotate indicates to the brain how near or far an object is, with nearer objects requiring a greater degree of inward rotation than objects farther from the face. To converge, the near triad occurs, the eyes converge, accommodation is activated, and the pupils constrict.

“Contact lens” is a lens placed on the exterior surface of the eye of a subject.

“Curvature” or “radius of curvature” of a lens is generally measured in millimeters (mm) and referred to in terms of diopters or mm. When expressed in diopters, the curvature is determined with an appropriate refraction index. For example, for contact lenses, the refraction indices of air and tears would be taken into account along with the refraction index of the lens material in determining the curvature when expressed in diopters, while for spectacles only the indices for air and lens material would need to be used. For other lenses, such as thicker lenses or intraocular lenses, appropriate formulas and refractive indices can be used as known to those of skill in the art. Curvatures can be determined by a topographer device or radius scope using appropriate index information.

“Defocus” refers to a translation of a focal point along the optical axis away from a detection surface, such as a retinal surface. In general, defocus reduces the sharpness and contrast of an image. What should be sharp, high-contrast edges in a scene become gradual transitions, and fine detail in a scene is blurred or even becomes invisible.

The “DEM™ test” is the Developmental Eye Movement Test, which incorporates a sub-test of number calling in a vertical array and provides the means to evaluate oculomotor function with numbers in a horizontal array. The DEM™ test was developed by Dr. Jack Richman, OD and Dr. Ralph Garzia, OD.

“Diopter” (D) refers to unit of refractive power that is equal to the reciprocal of the focal length (in meters) of a given lens or portion of a lens.

“Divergence” is the opposite of convergence and is the ability to turn the two eyes of a subject outwardly to look at a distant object. This skill is needed for distance activities such as reading the board at school, driving, and watching TV. To diverge, the opposite of the near triad must occur, i.e., the eyes diverge, accommodation is inhibited, and the pupils slightly dilate.

“E-value” refers to a measure of corneal eccentricity, with a value of zero indicating a perfectly spherical cornea. A negative e-value indicates a flat central zone with a steep mid-periphery (an oblate surface), while a positive e-value indicates a steep center which flattens radially outwardly (a prolate surface).

“Fixation disparity” is a tendency of the eyes to drift in the direction of heterophoria. Heterophoria or latent squint is defined as a condition in which eyes in the primary position or in their movement are maintained on a fixation point under stress only and refers to a fusion-free vergence state. Fixation disparity refers to a small misalignment in which an image drifts slightly from the corresponding points but is still within the fovea with normal fusion and binocular vision. The misalignment may be vertical, horizontal or both. While strabismus prevents binocular vision, fixation disparity keeps binocular vision, however it may reduce a patient's level of stereopsis and cause asthenopia.

“Focus” is a point at which light rays originating from an object or direction converge, such as by refraction.

The “fovea” is a part of the eye, located in the center of the macula region of the retina. The fovea is responsible for sharp central vision, which is necessary in humans for reading, watching television or movies, driving, and any activity where visual detail is of primary importance. The human fovea has a diameter of about 1.2 mm-1.5 mm and subtends a visual angle of about 4-5 degrees (2-2.5° to each side of the optical or visual axis). The best correctable vision (BCVA) is about 20/20.

The “front surface” of a lens refers to the surface through which light enters the lens in its normal and intended usage. For example, for a contact lens the front surface is the surface facing outwardly when worn on the eye of a subject, in contact with the air.

“Horopter” is the locus of points in space that have the same disparity as fixation. This can be defined theoretically as the points in space which project on corresponding points in the two retinas of a subject, that is, on anatomically identical points. A “theoretical horopter” or “Veith-Muller Circle” means a theoretical geometric circle passing through the optic centers of two eyes by which points adjacent to the point of fixation, both lying on the circle, theoretically fall on corresponding retinal points. The theoretical horopter must be a circle passing through the fixation-point and the nodal point of the two eyes. An “empirical Horopter” is an experimentally determined ellipse passing through the optical centers of two eyes by which points adjacent to the point of fixation, both lying on the ellipse, are perceived to be stimulating corresponding retinal points. The shape of the empirical horopter deviates from the theoretical horopter.

“Image shell” refers to the generally concave area of clear focus created by the ocular refraction system wearing correction lenses (contact lenses or spectacles).

“Intraocular lens” (IOL) is a lens implanted in the eye, which may replace the eye's crystalline lens or coexist with it.

“Lag of accommodation or accommodative lag” is the difference between accommodative stimulus (+2.50 D at 40 cm target) and the accommodative response (focusing) that the stimulus is closer than the response. Lag of accommodation=(Accommodative stimulus−accommodative response) that is positive in value.

“Lead of accommodation or accommodative lead” is the difference between accommodative stimulus and the accommodative response that the response is closer than the stimulus. Lead of accommodation=(Accommodative stimulus−accommodative response) that is negative in value.

“Lens” refers to an optical element which converges or diverges light, in particular to a device which is not a tissue or organ of a subject.

“Macular sparing” is visual field loss that preserves vision in the center of the visual field, otherwise known as the macula. It appears in people with damage to one hemisphere of their visual cortex, and occurs simultaneously with bilateral homonymous hemianopia or homonymous quadrantanopia. Macular sparing can be determined with visual field testing. The macula is defined as an area of approximately ±8 degrees around the center of the visual field. Vision in an area of greater than 3 degrees must be preserved for a patient to be considered to have macular sparing because there is involuntary eye movement within 1 to 2 degrees.

“Macular splitting” is the opposing effect of “Macular sparing”, where vision in half of the center of the visual field is lost.

“Meridian” generally refers to a hypothetical line which extends along a curved surface of a lens. A “horizontal meridian” is a line which extends through a plane that is parallel to a surface supporting a user of the lens, such as a floor or the ground, when the user is wearing the lens. A “vertical meridian” is a line which extends through a plane that is perpendicular to the horizonal meridian and which is generally parallel to the sagittal plane of a user wearing the lens.

“NSUCO Oculomotor test” is an approach to assessing fine visuomotor skills test developed by NSUCO (Nova Southeastern University College of Optometry) by an experienced clinician. Only minimal equipment is required for administration of this test.

“Ocular defocus” refers to the image foci of an eyeball being gradually defocussed forward (shorter focus, that is, myopic ocular defocus) or backward (longer focus, that is, hyperopic ocular defocus), centrally outward from the Para-fovea to a peripheral portion of the retina. The forward (myopic) or backward (hyperopic) defocus is a combination of the optical system and the eyeball shape. The hyperopic ocular defocus having a shorter peripheral retinal shell is more common in myopic eyes. The myopic ocular defocus having a longer peripheral retinal shell is more common in hyperopic eyes.

“Oculomotor dysfunction” is a problem in the oculomotor system, having one or more problems of fixation, saccadic eye movement, and/or pursuit eye movement. This dysfunction prevents efficient reading skills and can also limit or reduce reading comprehension.

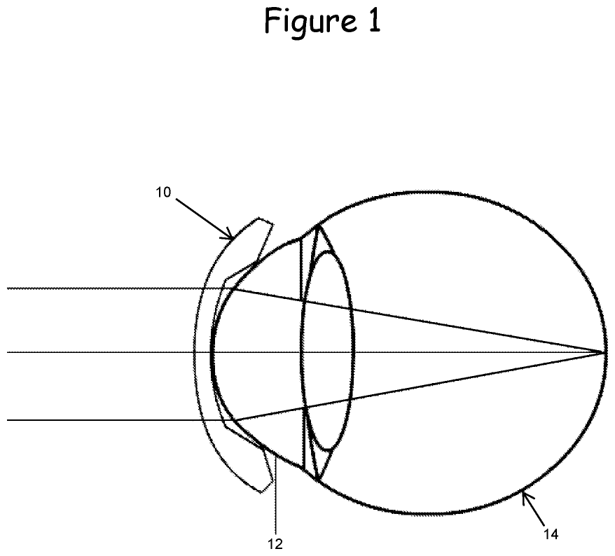

“On-axis,” when referring to light passing through a lens, refers to a direction substantially parallel to the optical axis of the lens. When light from an object enters a lens from a direction lying substantially on or parallel to the optical axis, the object is called a central object and the image formed by the lens is called a central image. In an ocular visual system, the on-axis image is conjugated to the fovea portion of the retina (as shown in FIG. 1).

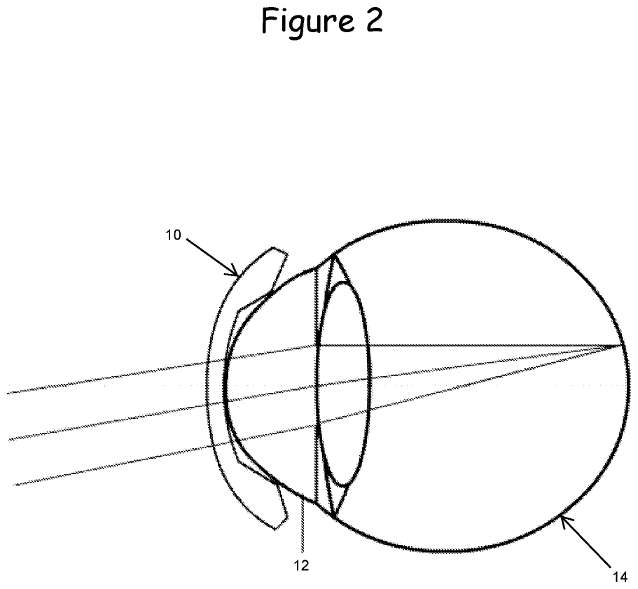

“Off-axis,” when referring to light passing through a lens, refers to a direction that is not substantially parallel to the optical axis of the lens, such that incoming light entering the lens deviates from the optical axis with an angle larger than zero. In an ocular visual system, an off-axis image is conjugated to the retinal areas out of the fovea portion of the retina, in particular in the parafovea or perifovea areas (as illustrated in FIG. 2). An off-axis can be further defined as “para-axis” if the incoming light enters the optical device deviating from the optical axis of the system with an angle beyond 2 degrees and within 10 degrees.

The “optical axis” in an optical device, such as a lens, means a line along which there is some degree of rotational symmetry, such that the device is radially symmetrical around the line.

“P-value” (p) is a quantity derived from the e-value (e) by the equation p=1−SIGN(e) e², where SIGN indicates the plus or minus value which is the same as that of the e value, i.e., if e<0, SIGN(e)=−1, and if e>0, SIGN(e)=+1. The p-value of a prolate cornea surface is less than 1.0 and that of an oblate cornea is higher than 1.0. The p-value of a perfect spherical cornea is 1.

“Panum's fusion area” means the region in front or back of the horopter in which binocular singleness of vision is present. It is narrowest at the fixation point and becomes broader in the periphery (see FIG. 11). Inside Panum's area, including the horopter, single images are viewed by functionally normal eyes, whereas outside Panum's area, either in front or behind, double images are seen. All objects outside Panum's area give rise to diplopia (double vision).

The “parafovea” is the intermediate area radially outward to a distance of 0.5 mm to each side and circumscribing the central fovea, where the ganglion cell layer is composed of more than five rows of cells, as well as the highest density of cones. The outmost parafovea zone subtends a visual angle of about 8-10 degrees (4-5° to each side of the optical or visual axis). The best correctable vision (BCVA) in this zone can be 20/50 (0.4 logMAR) up to less than 20/20 (0 logMAR).

The “perifovea” is the outermost region of macula found 1.5 mm to each side and circumscribing the parafovea, where the ganglion cell layer contains two to four rows of cells, and is where visual acuity is below the optimum. The outermost perifovea zone subtends a visual angle of about 18-20 degrees (9-10° to each side of the optical or visual axis). The best correctable vision (BCVA) in this zone is between 20/50 (0.4 logMAR) and 20/100 (0.7 logMAR).

“Peripheral refraction” of the eye can be measured with wide view (or open field) refractometers such as Shin-Nippon NVision-K 5001 or Grand Seiko WR-5100K, which has Wide-View window and allows subjects to relax during measurement, by looking into the window naturally with both eyes and fixating at any distances and directions.

“Phoria” is a misalignment of the eyes that only appears when binocular viewing is broken and the two eyes are no longer looking at the same object. The misalignment of the eyes starts to appear when a person is tired, therefore it is not present all of the time. A phoria can be diagnosed by conducting the cover/cover test.

The “ReadAlyzer/Visagraph” is a hardware and software package. It includes goggles that are fit precisely onto the patient's face, and these are used to scan micro-movements of the eyes as they target different visual signals on test pages. The goggles attach to a computer running the software, which provides data analysis, display, and storage.

“Refractive power” or “power” is the degree to which a lens converges (or diverges) light. The power of a lens is equal to the reciprocal of its focal length in meters, or D=1/f, where D is the power in diopters and f is the focal length in meters. “Plus power” refers to an extent of convergence of light, to bring a near object into focus with a lens, while “minus power” refers to an extent of divergence of light, to bring a far object into focus.

“Retinal correspondence” is either normal retinal correspondence (NRC) or abnormal retinal correspondence (ARC). NRC is a binocular condition in which both foveas work together as corresponding retinal points, with resultant images fused in the occipital cortex of the brain. ARC is binocular sensory adaptation to compensate for strabismus. The fovea of the non-deviated eye and non-foveal (usually parafovea) point of the deviated eye work together, permitting binocular fused singleness of vision.

“Rigid contact lens” refers to one whose surface does not change shape so as to assume the contour of a corneal surface when placed on an eye. Rigid contact lenses are typically made from PMMA [poly(methyl methacrylate)] or from gas-permeable materials such as silicone acrylates, fluoro/silicone acrylates, and cellulose acetate butyrate, whose main polymer molecules generally do not absorb or attract water.

“SCCO oculomotor test” is an approach to assessing fine oculomotor skills developed by SCCO (South California College of Optometry) which is performed by an experienced clinician. The test is scored by +1 to +4 for fixation maintenance, pursuit and saccade.

A “Shack-Hartmann system” can be used to measure eye lens aberrations. A high-resolution visual display displays spots that the user views through a lenslet array. The user then manually shifts the displayed spots (i.e., the generated wavefront) until the spots align. The magnitude of this shift provides data to estimate the first-order parameters such as radius of curvature and hence error due to peripheral defocus and spherical aberration.

A “soft contact lens” is one that is formed from a material whose surface generally assumes the contour of a corneal surface when placed onto a cornea. Soft contact lenses are typically made from materials such as HEMA (hydroxyethylmethacrylate) or silicone hydrogel polymers, which contain about 20-70% water.

“Spectacles” or “eyeglasses” refer to a frame with lenses which is worn in front of the eyes. The frame is normally supported on the bridge of the nose and by arms placed over the ears.

“Spherical aberration” refers to the deviation of a device or portion thereof from the focus of a perfect lens which focuses all incoming rays to a point on the optic axis.

“Stereopsis” means perception of depth on the basis of visual information deriving from two eyes. Human eyes are located at different lateral positions on the head resulting in two slightly different images, mainly in the relative horizontal position of objects, projected to the fovea of the eye. The positional differences are referred to as image disparities and are processed in the visual cortex to yield depth perception. Binocular fusion is required for stereopsis but not vice versa.

“Suppression” as used herein refers to an inhibitory mechanism producing total or partial cancellation of one of the two monocular images observed by two eyes. The adaptive value of this mechanism is to avoid confusion or diplopia. “Intermittent central suppression (ICS)” is a defect in normal binocular vision that causes a repetitive intermittent loss of visual sensation in the central area of vision with the central vision of either eye “turns on and off”, while peripheral fusion is maintained. The monocular suppressions during binocular fusion are characterized by brief cycles of suppression limited to the central 2-3 degrees. Monocular suppression may last about 2-3 seconds followed by binocular fusion for 2-3 seconds, and the same or fellow eye may be suppressed for 2-3 seconds. The ICS may happen in one eye as constant ICS or alternate in two eyes as alternate ICS.

“Translating” bifocal or multi-focal contact lenses are lenses that have at least two separate areas or zones for far and near vision respectively.

“Tropia” is a misalignment of the eyes that is always present. Even when the eyes are both open and trying to work together, large angle misalignments are apparent. A tropia is the resting position that eyes go to when covered or when fusion is broken by repetitively and alternately covering each eye.

“Visual acuity” refers to the clarity of focus provided by a particular optical system (e.g., a lens and/or a cornea of an eye).

The “visual angle” is the angle that light subtends with respect to the visual or optical axis, preferably measured from the principal plane.

The “visual axis” means a straight line extending from a viewed object through the center of the pupil of a subject to the fovea area of the retina in a human eye.

“Visual efficiency” is the eye's ability to track, converge, and focus quickly. Evaluation of visual efficiency skills is composed of four systems including, the oculomotor system, the accommodative system, the vergence system and the sensory system. Visual efficiency is needed for proper visual processing of visual information. Having a difficulty in this area is generally referred to as a visual efficiency problem.

“Vergence dysfunction” is a problem of the vergence system, for the simultaneous movement of both eyes in opposite directions, to obtain or maintain single binocular vision as an object's distance varies. There are several types of vergence dysfunction involving one of the following diagnoses: (1) Fusional vergence dysfunction, (2) Convergence insufficiency (CI), (3) Basic exophoria, (4) Divergence excess, (5) Convergence excess (CE), (6) Basic esophoria, and (7) Divergence insufficiency.

“Visual information processing skills” is a term that is used to refer to the brain's ability to use and interpret visual information from the world around an individual. The process of converting light energy into a meaningful image is a complex process that is facilitated by numerous brain structures and higher-level cognitive processes.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Equations

The following equations can be used to determine the e-values of the horizontal and vertical meridians of a lens:
1. Rₚ is the radius of curvature at peripheral distance “d”; R_{c} is the radius of curvature at center; d is the distance of a peripheral point to the lens center. E is eccentricity; p is the p-value.

$$E = SIGN ( {R}_{p} - {R}_{c} ) * SQRT ( ABS ( {R}_{p}^{2} - {R}_{c}^{2} ) ) / d \qquad ( Equation 1.1 )$$

$$p = 1 - SIGN ( E ) * {E}^{2} \qquad ( Equation 1.2 )$$

$${R}_{p} = SQRT ( {R}_{c} + SIGN ( E ) * {d}^{2} {e}^{2} ) \qquad ( Equation 1.3 )$$

2. The uneven rotationally aspheric ADF zone, along axis X^{o} can be formulated as: En is the predetermined horizontal (0-180 degree) e-value; Eᵥ is the predetermined vertical (90-270 degree) e-value; HRₚ is the radius of curvature at the point radially outward along horizontal axis for a distance “d”; VRₚ is the radius of curvature at the point radially outward along vertical axis for a distance “d”; XRₚ is the radius of curvature at the point radially outward along axis X^{o} for a distance “d”; Eₓ is the e-value of the axis X^{o};

Apply EQUATION 1.3 to calculate HRₚ and VRₚ:

$${HR}_{p} = SQRT ( {R}_{c} + SIGN ( {E}_{h} ) * {d}^{2} {E}_{h}^{2} ) \\ {VR}_{p} = SQRT ( {R}_{c} + SIGN ( {E}_{v} ) * {d}^{2} {E}_{v}^{2} ) \\ {XR}_{p} = {HR}_{p} + {sin ( {X}^{o} )}^{2} * ( {VR}_{p} - {HR}_{p} ) \qquad ( Equation 2.1 )$$

Apply EQUATION 1.1 to derive Eₓ:

$${E}_{x} = SIGN ( {XR}_{p} - {R}_{c} ) * {( ABS ( {XR}_{p}^{2} - {R}_{c}^{2} ) )}^{1 / 2} / d , \qquad ( Equation 2.2 )$$

### Visual Efficiency

Visual efficiency is the eye's ability to track, converge, and focus quickly. Visual efficiency is needed for proper visual processing of visual information. The visual acuity, refractive error, ocular motility, accommodation and binocular vision are all together important contributors to visual efficiency. Abnormalities or dysfunctions in one or more of the aforementioned functions may influence visual attention and induce learning-related vision problems. Visual efficiency problems, especially binocular dysfunction such as accommodation insufficiency (AI) or convergence insufficiency (CI), may cause eyestrain or asthenopia that could be one of the reasons of rapid myopia progression.

There has been an enormous amount of research about the relationship of visual efficiency and read-to-learn performance. The most well-known case analysis system for visual efficiency is Optometric Extension Program (OEP-21-points) analysis, of which the tests are sorted by number codes. There are some other analyses and clinical criteria proposed to help making decisions. The OEP analysis tests refraction, phoria deviation, the vergence system, and the accommodation system, in both near and far distance. A comprehensive evaluation should also include checking the oculomotor and sensory systems.

The skills to measure visual efficiency are well known for well-trained eye care practitioners (ECP), especially behavioral optometrists or eye doctors (OD/OMD). The most important goal of visual efficiency in humans is to maintain the spatial relationship for the two eyes aligned and focused to form a single, fused clear image in all distances, near and far. The diagnosis and categorization of a visual efficiency abnormality or dysfunction is usually made by comparison of the measured functional data against normative clinical data of corresponding items. The relative size or range among some of the measured items also needs cross checking with the clinical criteria evolved from graphical analysis of the OEP-21-points to assist in quickly assessing whether the vergence or accommodative disorders exist. The accepted normative clinical data and criteria for cross checking of visual efficiency, including but not limited to the Sheard's criteria and Pervical's criteria, are well known to the skillful ECPs.

Besides checking the accommodation and vergence data to assess whether there is a possible visual efficiency problem, an evaluation of the sensory system can gauge the quality of binocular vision more directly. Clinically, eye care professionals usually assess the sensory system by checking binocular fusion, fixation disparity, retinal correspondence (ARC or NRC), suppression, intermittent central suppression (ICS), and three-dimensional stercopsis.

Tropia or strabismus is a condition in which the eyes do not properly align with each other when looking at an object. The condition may be present intermittently or constantly. The eye that fixates on an object can alternate between eyes or can be constant in one eye. The deviation may be present intermittently or persistently. Tropia is a significant binocular abnormality but usually does not display significant visual efficiency problems. The sensory system of a tropia usually adapts by suppression or ARC to eliminate confusion for no or little interference with reading tasks.

On the other hand, nonstrabismic visual efficiency problems are nonobvious without significant eye deviation, while they can compromise reading comprehension and school performance profoundly. The nonobvious binocular dysfunctions are usually neglected or misdiagnosed as attention deficit disorders (ADD/ADHD) or dyslexia and miss the chance of early detection and remediation.

Experienced ECPs can diagnose nonstrabismic visual efficiency problems by performing a comprehensive visual examination, which includes checking ocular health and visual acuity with refraction, and an evaluation of visual efficiency skills for the oculomotor system, accommodative system, vergence system and sensory system. For some developmentally delayed children, their visual processing skills can be probed to discover a perceptual skill deficiency. If a nonstrabismic visual efficiency problem is discovered, the child may need to consult a behavioral optometrist or an ophthalmological specialist for further management. The therapeutic strategy can include prescribing a pair of spectacles or contact lenses for correction of the refractive errors, accommodation problems, and/or compensation of the phoria deviation angles. The refractive errors and accommodative problems may need plus or minus spherical powers for correction or compensation of the defect, while the deviation angle of the phoria or the vergence dysfunction can be assisted or compensated with spherical or prism lenses. Professionals can determine the spherical or prism powers by manipulating the measured phoria angles, accommodation powers, vergence ranges and most importantly the type of visual efficiency problem that is determined in the comprehensive exam. The spherical or prism powers determined for compensation may work only temporarily or within limited distance such as only for an arm's length. That is because the compensating power provided is usually for relieving the visual stress but not to improve or remediate the efficiency problems. The required compensation spherical or prism powers are usually different for far and near distance. The eyes may adapt to the new deviation angle, for less demand in accommodation or vergence powers with the assistance of the compensating spectacles, and worsen in the dysfunction with time.

Vision therapy (Visual training or “VT”) is the treatment of choice for the patient identified as inattentive due to visual efficiency problems, especially those associated reading/learning difficulties and behavior concerns. This therapy can be defined as utilizing behavior modification and biofeedback designed to rearrange conditions allowing for new insights and an alternative approach fostering efficient, effective and, ultimately, effortless eye teaming. VT will need office and home training courses. It usually takes at least 3 months to get improvement. The learned skills may regress and need booster training periodically after completion of the initial training course.

The compensatory spherical or prism spectacles or contact lenses can assist visual efficiency through improvement of binocular image clearness for better central fusion at central fovea area of the retina, which is regarded as the major area of the sensory system to have a fused clear binocular vision. A plus spherical power is usually added to assist the accommodation system for clearer near vision at arm's distance, such as that of presbyopia or accommodation insufficiency (AI). The added minus or plus spherical power may also change the vergence system by altering accommodative convergence to improve the deviation angle and binocular fusion range. If adding spherical power in spectacles to improve the visual efficiency problems, the wearer may need two pairs of spectacles one for near and one for far distances. Alternatively, the patient can use a pair of bifocal or multifocal glasses or contact lenses for daily work or school.

The compensatory prism is usually incorporated into spectacles to assist the vergence system with excessive deviation angles at far or near distance, such as that of a fusional vergence dysfunction or convergence insufficiency (CI). The prism power is not prescribed to improve the vergence system or to straighten the eyes, while instead it is added to move the whole image horizontally or vertically into the central fovea of the retina of the deviated eyes for binocular images to promote fusion effortlessly. The required relieving prism powers are usually different at far and near distance so that the patient may need to replace the spectacles when looking in different distances. It would be very inconvenient for daily life especially in school. The relieving prism does not remediate the phoria deviation nor improve the vergence system, but satisfies the sensory system for better central fusion at certain distances. The phoria angle may enlarge with gradual adaptation to the added prisms and recurrence of the dysfunction and asthenopia may happen, requiring more prisms to relieve the symptoms.

Spherical and prism spectacles can be incorporated to improve the sensory system and allow the central fovea images to fuse more effortlessly. While Visual training (VT) can extend the fusional range for the extraocular muscles to sustain central fusion without image breakage. In other words, VT is to train the extraocular muscles and brain control to compensate for the phoria angle for better sustaining of binocular fovea fusion like a prism may do, while VT usually does not improve the phoria angle. The improved conditioning after successful VT may regress if not boosted periodically and the sensory system may adapt abnormally by itself with some form of suppression to relieve eyestrain and confusion.

Patients having visual efficiency problems may adapt to the dysfunction in several ways if not discovered and addressed properly. The most common adaptation for children with visual efficiency problems is to avoid near tasks, which may be regarded as attention deficit (ADD/ADHD) or dyslexia in school. Another adaptation is to suppress one eye and see with one eye at a time. The phenomenon is involuntary and may suppress constantly in one eye or alternating between fellow eyes.

Myopia may also progress more rapidly as an adaptation to the visual efficiency problems since higher myopia may relieve strenuous convergence and/or accommodation requirements at arm's distance for near tasks. If the suppression happens constantly in one eye, the myopia in the sighting eye (non-suppression eye) for near tasks may progress faster than its fellow eye and become anisometropic. If the more myopic eye sees much clearer than its fellow eye in doing near tasks, developing anisometropia may avoid double vision in a case involving visual efficiency problems. Hence, myopia and anisometropia can be an adaptation for visual efficiency problems. If we neglect the underlying visual efficiency problems and only compensate for the induced errors such as myopia or anisometropia, the induced errors will worsen rapidly to degrade the compensation power for a new adaptation. It is not uncommon that myopia with convergence insufficiency (CI) may progress rapidly since children cannot avoid heavy near tasks in school. The myopia is an adaptation of the strenuous convergence and accommodation demand of AI or CI cases, while a pair of new spectacles for far vision breaks the adaptation and then myopia has to progress further to set up a new adaptation for less strenuous visual demand at arm's distance. The same condition for the CI case results in an adaptation with anisometropia, of which the more myopic eye is usually used for near tasks, and reading is accomplished with only one eye, for less strenuous convergence demand. If a pair of spectacles is prescribed to fully correct the anisometropia without remediation of the CI, the anisometropia may increase to achieve a new adaptation for monocular vision that requires less effort for the CI patient to read and work at arm's distance. For those CI cases, the myopia and anisometropia may progress rapidly with heavier near work if the underlying visual efficiency problems have not been discovered and remediated properly.

Currently, binocular dysfunction is analyzed by challenging the accommodative and vergence systems with spherical and/or prism lenses to determine the power ranges and compensate for the defect with the spherical and/or prism lenses. The visual training is a conditioning training (VT) to teach patients to perceive one stimulus and signal the occurrence of a proper reaction. We still do not know the physiological or anatomical origin that can be remediated directly for a permanent cure of most visual efficiency problems. The most common vergence system problem is convergence insufficiency (CI) having a prevalence rate of 2-17% with an average of 13%, with a low AC/A ratio, high exophoria at near distance, a reduced positive (convergence) vergence range and possibly accommodative insufficiency (AI). When prescribing spectacles for a CI patient, adding plus power to compensate for accommodation lag for assisting near vision, may drive the eyes outward and increase exophoria at near distances. The AC/A ratio of CI is usually lower than normal (3/1 −6/1).

The foregoing binocular evaluations, compensation lenses and VT for visual efficiency problems all aim to correct the fusion or motor ranges of central fovea images. It is a new discovery that the plus power added only to the peripheral portion of contact lenses, can reduce exophoria at near distance immediately and significantly, while also simultaneously diminishing accommodation lag at near distance, without compromising far vision. That is to say, we can justify the spatial relationship for all distances to remediate visual efficiency problems by manipulating the peripheral ocular defocus of the image, which has never been taught in traditional concepts about eye teaming.

It is yet another discovery to use the contact lenses mentioned above for the convergence excess (CE) condition to improve esophoria (inward deviation) at near distance. CE is characterized with high AC/A, higher esophoria and reduced negative (divergence) fusion range at near distance, which is almost the opposite condition of CI in traditional categorization for diagnosis and remediation. It is ironic that the same central distant (CD) multifocal lenses can also restore the spatial relationship of the CE patients instantly as effective as that for the CI patients. The sensory system can improve dramatically, in both CI and CE conditions, with reduction or elimination of fixation disparity and associated phoria for less or no prism powers required for binocular fusion.

In order to use a CD multifocal lens to treat both CI and CE conditions, we need to know how the multifocal lenses alter the sensory system for binocular fusion at the peripheral portion of the eyes. To understand binocular fusion, we need to know the Panum's fusion area for singleness of vision that is defined by theoretical and empirical horopters. Binocular fusion occurs only in a small portion of visual space around where the eyes are fixating. Running through the fixation point in the horizontal plane is a curved line called the empirical horizontal horopter. There is also an empirical vertical horopter tilted away from the eyes above the fixation point and towards the eyes below the fixation point. The horizontal and vertical horopters mark the center of the volume of singleness of vision. Within this thin, curved volume, objects nearer and farther than the horopters are seen as single. The volume is known as Panum's fusion area and outside of Panum's fusion area, double vision occurs.

The traditional visual acuity and binocular fusion are defined for central fovea retina that has the best correctable vision for binocular fusion and stereopsis. It is true that if a person has strabismus and cannot fuse the binocular central images, there will be double vision, monocular central suppression or amblyopia that are attributed to the central fovea area for diagnosis and management. The resolution of the peripheral retina is generally much lower with much worse correctable vision. It is also difficult to qualify or quantify the peripheral binocular fusion, hence the peripheral visual field is usually neglected and regarded as unimportant for binocular fusion. However, the central visual field is only 1% of the total visual field despite having very sharp 20/20 or better acuity, so that the peripheral 99% visual field and its fusion may play an important role in maintenance of binocular fusion for eye teaming. The model of the horopter and Panum's fusion area offers the best support for the importance of peripheral fusion. The Panum's area is narrower at the fixation point (center fovea) and progressively wider to the periphery (peripheral retina). When we fixate a target at the center and fuse the binocular central images, we also need a binocular peripheral field to fuse as wide as possible for better visual efficiency. The arrangement of Panum's fusion area is an adaptation to facilitate peripheral fusion with the lower resolution of the peripheral retina. The oblique rays in the peripheral visual field enter the peripheral portion of the cornea to form an image at a peripheral portion of the retina. The oblique rays entering the peripheral portion of the cornea will induce oblique astigmatism, which is against the rule (ATR) astigmatism in horizontal meridian (J₀) and with the rule astigmatism in vertical meridian (J₉₀). That is how the peripheral retina perceives peripheral images for binocular fusion. Hence, the characteristic of image perception and binocular fusion at a peripheral portion of the retina is quite different from that of the central fovea, and it is more difficult to detect peripheral fusion with the techniques for detecting central fusion.

High quality fusion for an efficient sensory system should preferably have a clearest central visual field in the fovea, as well as the blurrier toric peripheral visual field in the peripheral retina, and both fall within the Panum's fusion area for binocular fusion. The horizontal empirical horopter is the key in binocular fusion. According to the Hering-Hillebrand deviation, the horizontal empirical horopter is flatter from theoretical horopter at short fixation distances and becomes convex for farther fixation distances. At short fixation distances, the horizontal empirical horopter is a concave parabola flatter that a circle. At some given distance, called the abathic distance, the empirical horopter becomes a straight line. Finally, for fixation distances farther than the abathic distance the empirical horopter is a convex parabola. In other words, the Panum's fusion area will be concave, straight, or convex in shape at different fixation planes.

The investigations of Panum's fusion area were usually approached geometrically including trigonometric analysis of the visual axis and angulation in space. While the accommodation or focusing system should not be overlooked since the horopter plane in space is determined with the convergence and the accommodation systems together to form a clear image for the whole retina in the horopter space. The width of the Panum's fusion area is also known as confusion of accommodation corresponds to the convergence angle of both eyeballs. While fixating a nearer point, the Panum's fusion area will become wider with a larger image disparity on the retina. That is to say, if the peripheral image shell is too blurry for peripheral image to fuse within the Panum's fusion area, the binocular fusion can be disrupted or compromised easily despite the central fusion being intact. The nearer viewing distance shall induce larger disparity that is more difficult for keeping fusion.

The macular area, substantially the central 4-5° degrees fovea area, projects to a different area in visual cortex of a brain, where is different from that of the peripheral retina and is the reason of eliciting macula sparing in a damage to the visual cortex. The phenomenon also tells us the central image and peripheral image can be regarded as two separated image shells for fusion. When testing the motor fusion with stereogram, we may be aware that the central image remains fused with stercopsis, while the peripheral image in outer box disrupted in advance of central image breakage. The central image shell is essential for clear binocular vision and stercopsis that requires accurate accommodation, sensory fusion and motor fusion. While the peripheral retina area, beyond the macula, forms the peripheral image shell that is important in allocation and stabilization of the binocular central fusion.

The shape of the eyeball, the optical system including the cornea and crystalline lens, and the retinal shell for forming images vary in the general population. The shape of the eyeball may change with physiological growth in young ages (usually before 7-9 y/o), of which the physiological eyeball growth expands nearly equally in anterior-posterior as well as equatorial dimension. While the myopic elongation usually expands along the anterior-posterior axis (axial elongation), with less and less elongation to the peripheral portion of the retina and little or nearly no elongation at equator. The axial elongation accompanying myopia progression may alter the peripheral image focus in peripheral retina more profoundly. The myopic axial elongation will end up with relative hyperopic ocular defocus, progressively increasing outward to the peripheral portion of the retina, if the myopia is corrected with regular single vision spectacles or contact lenses. The peripheral ocular defocus on a human eye, can be detected objectively using a commercially available auto-refractor (e.g., Shin-Nippon NVision-K 5001 or Grand Seiko WR-5100K, COAS Shack-Hartmann aberrometer) for mapping the focal points at the desired on-axis and off-axis visual angles. Investigations with MRI (Magnetic Resonance Imaging) have also discovered the peripheral hyperopic defocus is correlated with retina shell deformation as myopia progresses. The changes in retinal curvature in highly myopic eyes are more manifest in the horizontal meridian than the vertical meridian, particularly at the temporal retina, which may account for the significant hyperopic peripheral defocus in the image shell.

If an object falls outside of the Panum's fusion area of a person's fixation point, the image of the object will be doubling and unable to fuse, which is called physiological diplopia and may happen at the central or peripheral visual field. The Panum's fusion area is narrower at the center and becomes wider to the peripheral visual field, which corresponds to the lower resolution and against the rule astigmatic image at the peripheral portion of the retina. The peripheral retina has been adapted for fusing the blurrier and distorted image formed by the oblique rays entering the peripheral portion of the cornea. When we fixate binocularly at a near point for reading, the central fovea (4-5°) is used for fixation and perceives the codes, while the parafovea, perifovea and peripheral retina would help relocation and binocular fusion in saccadic eye movement. Saccadic eye movement is a rapid, conjugate, eye movement that shifts the center of gaze from one part of the visual field to another. Saccades are mainly used for orienting gaze towards an object of interest. The binocular vision for reading is not a static condition but a kinetic process in which the eye has to relocate quickly, and pause a few seconds to focus and fuse binocularly for a clear image, and thereby perceive the words repeatedly.

The traditional concept defines normal binocular vision in free space by coordination of the accommodation system for focusing at all distances and the vergence system for aiming at the object for binocular fusion, while both systems are regarded as only for integration of the fovea images. The fovea subtends only a 1-2° degree visual angle to each side of the visual axis, which is about 1% of the total visual field. Though the fovea is very important for clear vision with best resolution, it is not broad enough for maintenance of binocular fusion. It has to be the peripheral visual field that can fuse first for the central field to align precisely. The wider the image shell falls within the Panum's fusion area for fusion, the more accurate and efficient the central image would be able to fuse precisely for a clear central image. If a wide peripheral image focuses forward (shorter focus) or backward (longer focus) to the retinal shell and cannot satisfy the requirement of Panum's fusion area for fusion, the eyes will be forced to seek for a farther or closer fixation and focusing point to restore peripheral fusion and eliminate double vision or confusion. The farther or closer fixation distance required for peripheral fusion may alter at least one of the accommodative or vergence system and cause blurry of central image or disrupted/compromised central fusion. If the binocular central fusion is disrupted, there will be diplopia or suppression. If the central fusion is compromised but not disrupted, there will be ICS (intermittent central suppression), fixation disparity, or blurriness of the central image. The compromised images may still fall within Panum's fusion area for fusion but the image quality will be poorer, which can be detected as fixation disparity, ICS, accommodation lag or spasm in eye check.

That is to say, the mismatch of central and peripheral image shells may drive binocular re-fixation by the vergence and accommodation systems for a fusible status to prevent diplopia. The expense of the re-fixation is disturbance of the fovea image for poorer vision quality that may display fixation disparity, ICS or blurriness/fluctuation of vision. The poorer vision quality in turn may need frequent refocusing by the accommodation system, moving the head forward/backward, inducing oculomotor dysfunction, eye strain or rapid myopia progression, which are common symptoms of visual efficiency problems.

The deviation of the fixation/focusing point from the viewing target will be detected in traditional visual efficiency tests, such as the OEP-21 points exam, as abnormal exophoria/esophoria (#8 and #13), unsatisfied Sheard's/Percival's criteria with abnormal relative accommodation (#20-PRA or #21-NRA), relative vergence (#16A-PRV, #17A-NRV, #16B-PFV, #17B-NFV) or an increased accommodation lag (MEM retinoscopy, #14A-net and #15A-net with an unfused cross cylinder and #14B-net and #15B-net with fused cross cylinder tests). The deviation of the fixation/focusing point from the viewing target may also induce oculomotor dysfunction, which can be detected and evaluated with NSUCO test, SCCO test, DEM™, or ReadAlyzer/Visagraph.

In other words, this innovation proposes an anatomical-optical factor that the eyeball shape and optical system of the human eyes may cause visual efficiency problems, which can be measured and remediated by adjusting the peripheral ocular defocus forward or backward with an anti-defocus (ADF) optical device, to realign the image shell to match wider retinal shell for better whole field fusion. The anti-defocus (ADF) device better matches the requirement of Panum's fusion area centrally outward to the peripheral portion of the retina, and further facilitates better central fusion to reduce or eliminate the visual efficiency problems.

It is also a new innovation that myopia progression can be attributed to the same anatomical incompatibility that the peripheral image shell does not fit the retinal shell at arm's distance for reading. Without being bound by a particular theory, it may be that not only the optical stimulation controls human ocular growth as the prior art has stated, but binocular fusion incompatibility may be the main factor to force the eyeball to change in shape. The evidence is quite prevalent among children with CI that their myopia may progress very quickly, especially if the patient is forced to study without avoidance of near tasks for CI. Once we prescribe anti-defocus (ADF) contact lenses for them to wear, their CI conditions usually improve dramatically for better central fusion without a compensation prism or VT. The myopia progression may also stop or slow down quite significantly. The ADF lenses according to the present invention realign the image shell to the whole retinal shell for falling within the Panum's fusion area and improving binocular fusion with precise fixation on the target, which further facilitates central fusion and focusing that can be detected by the traditional visual efficiency tests as aforementioned. Once the visual efficiency problem is remediated and the eyestrain relieved, rapid myopia progression can be slowed down or ceased.

It is yet another innovation to use a series of ADF optical devices having stepwise or progressive peripheral forward or backward peripheral foci, to assess the extent of peripheral ocular defocus in a patient with visual efficiency problems and realign the image shells fitting into the Panum's fusion area to normalize the central fusion and focusing. The assessment of the peripheral ocular defocus also needs to be monitored while introducing the ADF optical devices. It is usually hard to detect peripheral ocular defocus and peripheral image shell misalignment directly. We can test the central fovea fusion or the visual efficiency performance once the image misalignment has been corrected by the ADF device, which can be done by repeating the OEP-21 points test, or more conveniently, by monitoring the fixation disparity or binocular fusion/stereopsis to detect central foveal fusion. The central fixation disparity will realign more properly, the phoria deviation may be reduced with improved relative vergence and/or relative accommodation as aforementioned in OEP-21 points exam. For candidates having oculomotor dysfunction, the improvement can be quantified by repeating the NSUCO test, SCCO test, DEM™, or ReadAlyzer/Visagraph before and after wearing the ADF optical device. The comparison is preferred to repeat after wearing the device several days or weeks, allowing time to elicit new balance with the new optical system. An ECP then may convert and incorporate the detected peripheral ocular defocus for a preferred spectacles or contact lenses.

There are several traditional ways to compensate for visual efficiency problems, for instance CI and CE, using spherical or prism lenses. Prism spectacles do not remediate phoria, but displace the binocular images outward or inward with a deviation angle that allows binocular fusion with less strenuous effort, which may relieve eyestrain for some period, but patients often adapt to the relieving prisms and deviate more to display a larger phoria with reappearance of symptoms and/or suppression. The spherical lenses may alter accommodation and adjust the phoria deviation through AC/A indirectly and improve central fusion. However, the spherical device may work only for a preset distance. If using it for vision outside of the preset distance, the vision would become blurrier or the binocular fusion breaks. In case the visual efficiency problem is proven due to misalignment of the image shells and retinal shells, the image shell can be modified with the peripheral ADF optical devices for better fitting into the Panum's fusion area in all distances. The traditional ways of visual training (VT) to remediate the visual efficiency skills may still help to further expand the fusion range and improve the fusion skills in sever candidates, while take less training hours or booster by using the ADF optical devices continuously after training.

The shape of the peripheral retina determines how the image shell matches the retina shell from center to periphery. The Panum's fusion area at horizontal meridian follows the Hering-Hillebrand deviation, which will be a concave shape for short fixation distance and a convex shape for far fixation distance. In other words, if the peripheral retinal shell has a relatively hyperopic ocular defocus, the fixation distance has to be farther away from the eye for the image shell to fall in Panum's fusion area for fusion. The convex Hering-Hillebrand deviation in far distance also indicates the retinal shape in axial myopia, namely that the retina shell has a progressively hyperopic defocus centrally outward. The vergence system for binocular fusion is driven by peripheral retinal shell, while the focusing or accommodation is driven by the fovea. We know in hyperopic conditions the focal length will be longer, while in myopic conditions the focal length is shorter. The hyperopic ocular defocus has a farther fixation distance from the eyes for binocular fusion in this invention, while myopic ocular defocus has a shorter fixation distance from the eyes for binocular fusion.

The Hering-Hillebrand deviation is concave at near distance and the concave Panum's fusion area also progressively wider toward the periphery, which may accept less hyperopic peripheral ocular defocus while more myopic peripheral ocular defocus at near distance. On the other hand, the Hering-Hillebrand deviation of the Panum's fusion area is convex at far distance and progressively wider to the periphery, which may accept more hyperopic peripheral ocular defocus at far distance.

### Visual Efficiency Remediation

Myopic eyes usually elongate along their anterior-posterior axis, having a longer axial length with the peripheral retina less elongated, which may form a hyperopic defocus with a blurrier peripheral image focusing posterior to the retina while the fovea image is in focus when corrected with a spherical lens. The peripheral ocular defocus cannot be adjusted with accommodation or a single vision optical lens since the fovea image will be altered and falling out of focus at the same time. The eyes may adapt to the peripheral ocular defocus with the vergence system by fixating binocularly at a farther distance (converge less) to seek for a farther but clearer peripheral image for better peripheral fusion. Fixating at a farther distance for horopter vision may not be perfect but allows better binocular fusion. The central and peripheral images may fall in the Panum's fusion area for fusion after refixation but shall be substantially mismatched to display “fixation disparity” (exoFD), “intermittent central suppression” (ICS) or excessive accommodation lag. This model also explains the characteristics of convergence insufficiency (CI) with low AC/A ratio, and excessive exophoria at near with accommodation lag (MEM, #14A and 14A-net, 15A, 14B and 14B-net, 15B in OEP-21 points exam.). The ocular shape suitable for this model is not limited to the myopic condition. The eyes can be one of emmetropic, hyperopic or myopic simply if the peripheral retinal shell has a relative hyperopic defocus for most peripheral image shells to focus more posteriorly to the retina and trigger less convergence to fixate at a farther distance for binocular fusion.

The opposite model explains convergence excess (CE) when the peripheral retinal shell has an excessive myopic defocus. Hyperopic eyes may have a shorter axial length and a relatively broader equator, which may form a myopic ocular defocus with a blurrier peripheral image focusing anterior to the retina while the fovea image is in focus, when corrected with a spherical lens. The ocular peripheral defocus cannot be adjusted with relaxation of the accommodation system or a single vision optical lens since the fovea image will be falling out of focus at the same time. The eyes may adapt to the peripheral ocular defocus with the vergence system by fixating binocularly at a nearer distance (converge more) to seek for a nearer but clearer peripheral image for better peripheral fusion. Fixating at a closer distance for horopter vision may not be perfect for vision but allows better binocular fusion. The central and peripheral images may fall in the Panum's fusion area for fusion after refixation but shall be substantially mismatched to display “fixation disparity” (esoFD), “intermittent central suppression” (ICS) or accommodation lead. This model also explains the characteristics of convergence excess (CE) with high AC/A ratio, and esophoria at near with excessive accommodation (MEM, #14A and 14A-net, 15A, 14B and 14B-net, 15B in OEP-21 points exam.). The ocular shape suitable for this model is not limited to the hyperopic condition. The eyes can be one of emmetropic, hyperopic or myopic simply if the peripheral retinal shell has a relatively myopic defocus for most peripheral image shells to focus anterior to the retina and trigger more convergence to fixate at a nearer distance for binocular fusion.

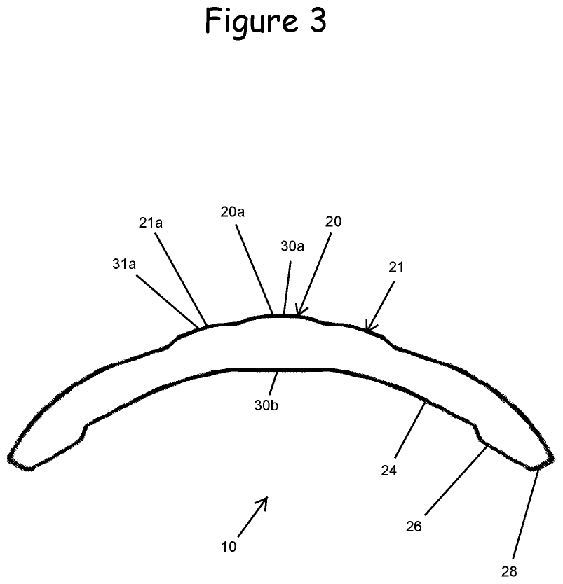

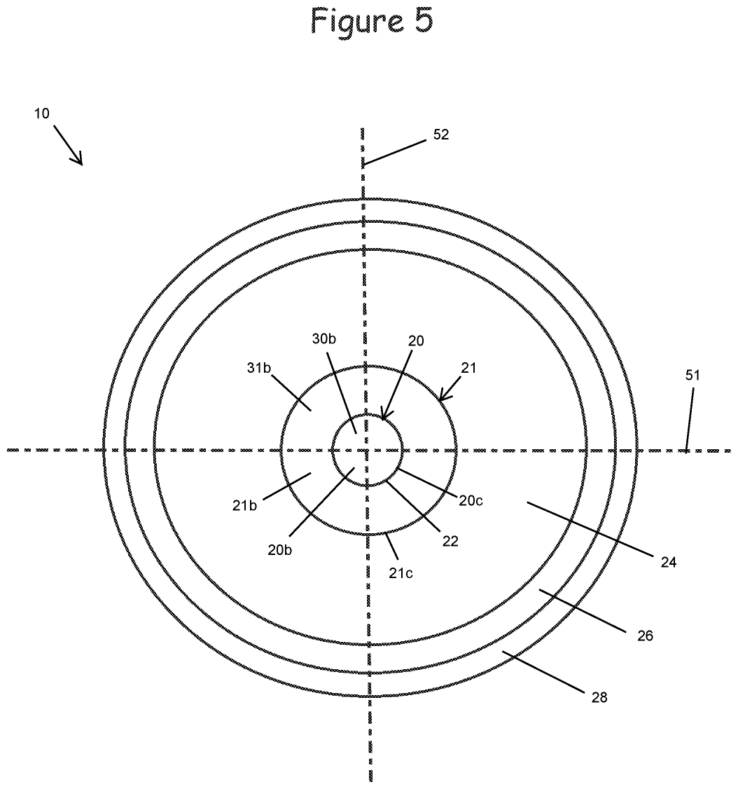

With reference to FIGS. 3-5, a peripheral anti-defocus (ADF) optical device, such as a contact lens, spectacle lens, or IOL, is provided with a center optical zone 20 having a refractive power for correcting distant vision, which subtends a visual angle of about central 4-5 degrees corresponding to the 1.5 mm fovea area located about 22.6 mm behind the corneal plane of a human eye. The adjacent outward portion of the optical device further provides an anti-defocus (ADF) zone 21 having a shorter or longer focal length to move the peripheral image shell forward or backward, respectively, which focuses images at the peripheral retina beyond the 4-5 degrees of the fovea far zone that corresponds to the parafovea (up to 10 degrees), perifovea (up to 20 degrees) and partial peripheral retina of the eye 14 at least up to 60 degrees.

The novel methodology of the invention can be used to design peripheral anti-defocus (ADF) devices for diagnosis and remediation of a visual efficiency problem and control of the associated myopia. The center power of the optical device for the fovea area of the eye 14 is always for correction of the distant vision. If the peripheral ADF zone 21 is more plus in power to bring the peripheral foci forward, it is called a hyperopic anti-defocus (H-ADF) lens. If the peripheral ADF zone 21 is more minus in power to bring the peripheral foci backward, it is called a myopic anti-defocus (M-ADF) lens. A H-ADF lens can be used to test and remediate the visual efficiency problems with peripheral retina that involves hyperopic ocular defocus, such as convergence insufficiency (CI). A M-ADF lens can be used to test and remediate the visual efficiency problems with a peripheral retina that involves myopic ocular defocus, such as convergence excess (CE). For other types of visual efficiency problems, the ECPs may test with either a M-ADF lens or a H-ADF lens to decide whether the problem is caused by image mismatch that can be remediated with a peripheral ADF lens.

A set of test lenses can be used to determine the anti-defocus power (ADP) for a lens according to the present invention. The testing set can be a series of spectacle lenses or contact lenses having a center optical zone 20 for far vision, with a diameter of 0.5-1.5 mm in a contact lens, or 1.5-4.0 mm in a spectacle lens. The center optical zone 20 can be assigned any spherical power that is convenient for clinical use for adding on powers to correct far vision. The most common choice is to apply “zero power” for the center optical zone 20, and an ADF zone 21 is adjacent to and progressively outward from the outer margin of the center optical zone 20 with a plus or minus power added for peripheral anti-defocus. The best mode of an ADF zone 21 is to make it progressively plus or minus in power, outward from the outer margin of the center optical zone 20. The progressive ADF zone of a spectacle lens for M-ADF lens can have a positive e-value (p-value<1) on the front surface, or a negative e-value (p-value>1) on the back surface. While that for H-ADF lens will be a negative e-value (p-value>1) on the front surface, or a positive e-value (p-value<1) on the back surface.

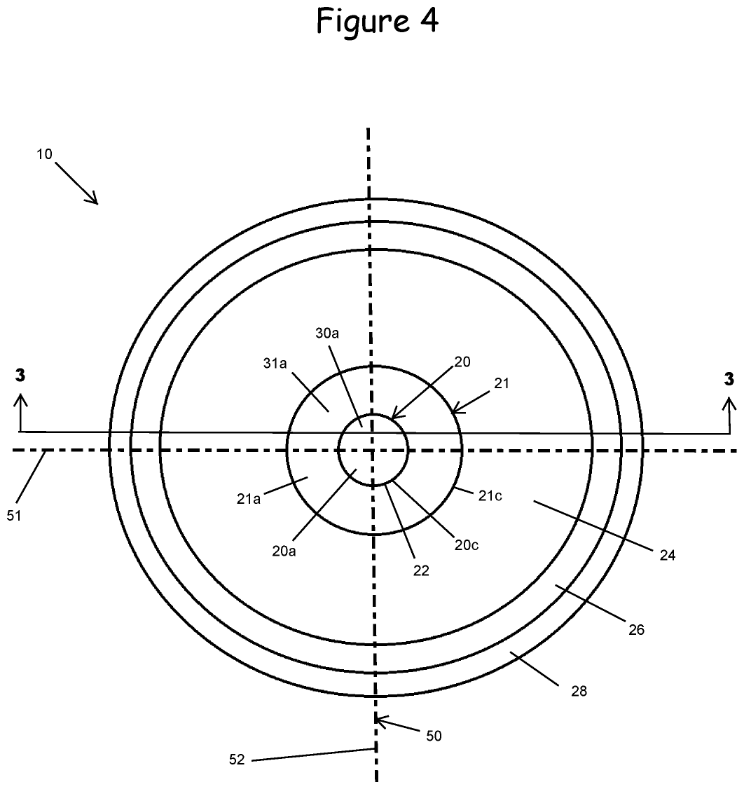

It is also possible to incorporate a progressive ADF zone 21 in a contact lens. Referring to FIG. 3 and FIG. 4, the progressive ADF zone 21 of a M-ADF contact lens shall have positive e-value (p-value<1) on the front surface ADF zone 21a with a curvature of 31a, or negative e-value (p-value>1) on the back surface ADF zone 21b. While the progressive ADF zone 21 of a H-ADF contact lens has negative e-value (p-value>1) on the front surface ADF zone 21a with a curvature of 31a; or a positive e-value (p-value<1) on the back surface ADF zone 21b. However, the progressive ADF zone 21a is preferably placed on the front surface of a contact lens 10 leaving the back surface for fitting the ocular surface. The contact lens 10 can be a soft contact lens, a rigid cornea lens (smaller than 12.4 mm) or a scleral lens (larger than 12.5 mm), while the soft contact lenses and scleral lenses are preferred for better centration with less movement on the eyes.

The visual angle, the field size and the image size should be ascertained in order to determine how the incoming light rays may form an image on retina shell for peripheral ocular defocus. The visual or optical angle conjugated to a principal plane can be calculated by the formula: θ=2*arctan (S/2D), where θ is the visual angle; S is the linear size of the object; and D is the distance from the object to the principal plane of the eye. For smaller angles, the image size or retina zone width conjugated to principal plane of a human eye can be figured by the formulas: image size I=[(2*π*d)*θ]/360, where d is the distance from the principal plane to the retina, and θ is the subtended visual angle of the object. Alternately, it can also be estimated by image size I=[2*(arctan (θ/2))*d]. The image or entrance field should be conjugated forward or backward from the theoretical principal plane located approximately 5.6 mm behind the front surface of the cornea apex or 17 mm in front of central retina for a 22.6 mm standard human eye. The axial length may elongate 1 mm for every −3 D myopia progression, which may also slightly increase the image size, while usually insignificant for designing the devices.

Furthermore, if the present optical device is a contact lens, the visual angle of the retina areas can be conjugated to a zone width on a contact lens or corneal plane that is located 22.6 mm in front of the fovea or 5.6 mm anterior to the principal plane. If the optical device is a spectacle lens, the visual angle can be conjugated to a zone width on spectacles that is positioned 12 mm in front of the cornea, 17.6 mm in front of the principal plane or 34.6 mm in front of the retina. To conjugate the zone width to visual angle, one degree is 1/360 of a circle, which is conjugated to the 17.5 mm zone in 1 meter distance, or the 7 mm zone in 40 cm reading distance, or 0.31 mm zone in spectacles distance that is 17.6 mm in front of principal plane, or 0.1 mm zone on cornea or contact lens surface that is 5.6 mm in front of the principal plane.

As shown in Table 1 below, the 4-5 degrees fovea is conjugated to a 0.5±0.1 mm zone on the contact lens plane or 1.55±0.2 mm zone on the spectacles plane. The 9-10 degree span of the parafovea then is conjugated to a 0.85±0.1 mm annular zone on the contact lens or 2.6±0.3 mm annular zone on the spectacles' plane. The 18-20 degree span of the perifovea is conjugated to a 1.8±0.2 mm annular zone on the contact lens plane or 5.5±0.5 mm annular zone on the spectacles' plane.

TABLE 1

| Locus to principal | | | | | | | Far | | |
|---|---|---|---|---|---|---|---|---|---|
| point (distance/ | Retina | Cornea | Spectacle | | Reading | | plane | | |
| zone width) | (17 mm) | (5.6 mm) | (17.6 mm) | | 40 cm | | 6M | | BCVA |
| Fovea 4-5° | 1.5 ± 0.1 mm | 0.5 ± 0.1 mm | 1.55 ± 0.2 | mm | 35 | mm | 527 | mm | 20/20 |
| Parafovea 9-10° | 2.5 ± 0.1 mm | 0.85 ± 0.1 mm | 2.6 ± 0.3 | mm | 58 | mm | 875 | mm | −20/50 |
| Perifovea 18-20° | 5.3 ± 0.4 mm | 1.8 ± 0.2 mm | 5.5 ± 0.5 | mm | 124 | mm | 1862 | mm | −20/100 |
| Peripheral[50-60° | 18 ± 1.0 mm | 6.0 ± 0.5 mm | 19 ± 1.0 | mm | 428 | mm | 6683 | mm | <20/200 |

The zone conjugated to the fovea area forms the center zone, while the annular zone conjugated to the parafovea and perifovea area and part of the peripheral portion of the retina forms the ADF zone on the optical device. The annular zone is not limited to being a round circle. It can be any shape that conjugates to each side of the visual or optical axis for the desired visual angle to have anti-defocus function. It is then very straightforward to design the ADF optical devices in testing the mismatch of the central and peripheral image shells, to determine the forward or backward direction of the ocular defocus and to quantify the anti-defocus power (ADP) required for remediation of the visual efficiency problems.

In a spectacle lens for testing hyperopic ocular defocus, the center optical zone is preferred to be zero power, with a diameter of 1.5-4.0 mm and an annular H-ADF zone 8-12 mm to each side of the center optical zone for a total diameter of 18-28 mm, for using at a vertex distance of 12-14 mm. The H-ADF zone 21 can be made progressively plus in power radially outwardly with a minus e-value (p-value>1) on the front surface, or a plus e-value (p-value<1) on the rear surface. The strength of the anti-defocus (ADP) can be controlled with gradient e-values between ±0.1 e-±2.0 e. The zero power for center optical zone 20 can be used to top on the patient's correction power for testing the peripheral ocular defocus without altering the correction power for far vision.

A spectacle lens for testing myopic ocular defocus, the center optical zone is preferred zero power, with a diameter 1.5-4.0 mm, and an annular M-ADF zone 8-12 mm to each side of the center optical zone for a total diameter of 18-28 mm, for using at a vertex distance of 12-14 mm. The M-ADF zone 21 can be made progressively minus in power radially outwardly with a plus e-value (p-value<1) on the front surface, or a minus e-value (p-value>1) on the rear surface. The strength of the anti-defocus power (ADP) can be controlled with gradient e-values between ±0.1 e-±2.0 e. The zero power for center optical zone can be used to top on the patient's correction power for testing the peripheral myopic ocular defocus without altering the correction power for far vision.

A contact lens for testing hyperopic ocular defocus, the center optical zone is preferred zero power, with a diameter of 0.5-1.0 mm and an annular H-ADF zone 3-4 mm to each side of the center optical zone for a total diameter of 6-10 mm center-ADF zone, for use on the cornea. The H-ADF zone 21 can be made progressively plus in power radially outwardly with a minus e-value (p-value>1) on the front surface for zone 21a; or a plus e-value (p-value<1) on the rear surface for zone 21b. While incorporating the ADF zone 21a on the front surface of a contact lens 10 is preferred to leave the rear surface only to fit on the corneal surface 12. The strength of the anti-defocus power (ADP) can be controlled with gradient e-values between +0.1 e-+3.0 e. The zero power for center optical zone 20 is convenient for over refraction with the trial frame without altering the correction power for far vision.

A contact lens for testing myopic ocular defocus, the center optical zone is preferred zero power, with a diameter of 0.5-1.0 mm and an annular M-ADF zone 3-4 mm to each side of the center optical zone for a total diameter of 6-10 mm center-ADF zones, for use on the cornea. The M-ADF zone 21 can be made progressively minus in power radially outwardly with a plus e-value (p-value<1) on the front surface 21a; or a minus e-value (p-value>1) on the rear surface 21b. When incorporating the ADF zone 21 on the front surface 21a of a contact lens 10, it is preferred to leave the back surface only to fit on the corneal surface 12. The strength of the anti-defocus power (ADP) can be controlled with gradient e-values between ±0.1 e-±3.0 e. Zero power for the center optical zone 20 is convenient for over refraction with the trial frame without altering the correction power for far vision.

The examination procedures to identify binocular dysfunction can be executed by trained ECPs to obtain required information for diagnosis. A checklist (Table 2) can be provided to guide the examination and record the baseline visual efficiency data before introducing the ADF lenses.

TABLE 2

1)PHORIA WITH OLD PRESCRIPTION
#3______(EX, ES) ( [symbol] )
#13A______(EX, ES) ( [symbol] )
2)RETINOSCOPY
#4(FAR WITH WORKING LENS +2.00D)
*OD: ______
OS: _____
#5(40 CM WITHOUT WORKING LENS)
*OD: ______
OS: ______
#6(1M WITHOUT WORKING LENS)
*OD: ______
OS: ______
MEM RETINOSCOPY (ACCOMODATION LAG)
OD: ______
OS: ______
3)SUBJECTIVE REFRACTION
#7(MENIFEST REFRACTION TO 20/20 OU)
OD: ______
OS: ______
4)INDUCED PHORIA
#8______(XP, EP) ( [symbol] )
#12______(BU, BD) ( [symbol] )
5)VERGENCE AT FAR (6M)
#9______(PRV, BO TO FIRST BLUR)
#10______/______(PFV, BO BREAK/RECOVER)
#11______/______(NFV, BI BREAK/RECOVER)
6)PHORIA AT NEAR & AC/A RATIO (−1D)
#13B______(XP, EP) (40 cm, CTL)
#18______(BU, BD) (40 cm)
AC/A______(XP, EP) (ADD −1D)
7)UNFUSED CROSS CYLINDER
#14A(UNFUSED JCC, DIM LITE)
*OD: ______OS: ______+C
#15A(#14A POWER, DIM LITE)
______(XP, EP)
8)FUSED CROSS CYLINDER
#14B(FUSED JCC, DIM LITE)
*OD: ______OS: ______+C
#15B(#14B POWER, LITED CROSS)
______(XP, EP)
9)VERGENCE AT NEAR (40 CM, CTL)
#16A______(PRV, BO TO BLUR OUT)
#16B______/______(PFV, BO BREAK/RECOVER)
#17A______(NRV, BI TO BLUR OUT)
#17B______/(NFV, BI BREAK/RECOVER)
10)ACCOMODATION POWER
BIOCULAR ACC. (33 CM, J4, ADD +2.50)
#19______D
MONOCULAR ACC. (40 CM, J4, ADD +2.50)
OD: ______OS: ______D
11)PRA (20/20, 40 cm, −TO BLUR OUT)
#20______D
12)NRA (20/20, 40 cm, +TO BLUR OUT)
#21______D
13)NPC (20/60, BREAK/RECOVER, FROM NOSE)
NPC: BR______RE______(CM)
14)NPA (20/60, BLUR/RECOVERY, FROM NOSE)
NPA: BL______RE______(CM)
15)ACCODATION FACILITY (±2.00D)
OD: 1 min______2 min
OS: 1 min______2 min
OU: 1 min______2 min
16)VERGENCE FACILITY (8 PRISMS BO/BI)
1 min______2 min______

SENSORY: 1. FD[ ]′ (EXO, ESO) AF[ ]Δ(BI, BO)
2. FVFD SLOPE[ ]
3. SUPPRESSION: FAR(W4D)[ ] NEAR[ ]

The vergence system of far and near dissociated phoria (#8, #13), fusion ranges (#10, #11, #16, #17), the accommodation system of accommodation lag (#14, #15), accommodation power (#19), accommodation facility, and the sensory system of fixation disparity, associated phoria or ICS, are the most important items for further evaluation of the anti-defocus effect. The sensory system is the most sensitive indicator in testing ADF lenses. The vergence system can be an alternative indicator if the sensory system does not display significant abnormality in initial exam. The accommodation system can be an adjunct indicator if the initial exam shows accommodation dysfunction that can be improved with the peripheral ADF devices.

The ADF testing lenses can be a series of spectacle lenses or contact lenses as aforementioned. The ADF spectacle lenses with gradient ADP can be made of loose trial lenses for use with a trial frame, or an accessary to be mounted on a phoropter or similar devices in a rotatory disk for quick operation. The accessary for mounting on a phoropter is preferred for easier comparison of the subtle change with a very short time lapse between replacing lenses, and ensures that the eyes see through the center optical zone of the ADF spectacles lenses, which is critical in testing peripheral ocular defocus. The ADF contact lenses with gradient ADP for testing can ensure that the center optical zone aligns on the visual axis, but the lapsing time for replacing contact lenses will be longer and less convenient. It is preferred to use ADF spectacle lenses for a quick survey and to apply the ADF contact lenses to confirm the anti-defocus strength that should be prescribed for improvement of the visual efficiency problems. It may take several days or weeks for the maximum effect to become manifest. The ECP may prescribe a pair of contact lenses, according to the ADP initially acquired with the testing devices as aforementioned, for use at home for at least 2 to 4 weeks and recheck the vergence, accommodation or sensory system, against the baseline visual efficiency data, to finetune the ADP for best remediation of the visual efficiency problems.

If the sensory system is available for an indicator, the procedures for checking ocular defocus will be as follows. Place the fixation disparity card or devices in a testing distance, the correction power of #7 or the #14A-net/#14B-net for presbyopia patient is applied as control, then introduce the ADF spectacle lenses (with zero power in a central zone) gradually from lower ADP (lower ±e-value) to higher ADP (higher ±e-value) top on the correction power and check the fixation disparity/associated phoria until it is a stable zero disparity or with maximum normalization. Finally, double check the binocular fusion/suppression at far distance to make sure the far vision is not compromised with the ADF lens, and record the raw ocular defocus strength so determined. If the vergence system is used as the indicator, the near dissociated phoria (#13) can be monitored while introducing the ADF lens continuously until #13 is reduced to a normal range and remove the vertical dissociation prism and recheck the fusion range (#16, #17) to check it against the residual phoria (#13) for the Sheard's or Percival's criteria. If the criteria are satisfied or improved significantly, double check the binocular fusion/suppression at far to make sure the far vision is not compromised with the ADF lens, and record the raw ocular defocus strength so obtained. The raw ocular defocus strength can be used to select a pair of testing ADF contact lens with zero power on the center optical zone to fine-tune the residual ocular defocus immediately in office. Alternatively, a pair of ADF spectacles or contact lenses can be prescribed and delivered based upon the raw ocular defocus strength and returned to clinic for fine-tuning the power and ADF strength in a few weeks. The procedure can also be repeated when a fine-tuning is required, if visual training (VT) is performed and has altered the accommodation or fusion range.

The peripheral anti-defocus devices for remediation can be a pair of contact lenses, spectacles lenses, orthokeratology lenses or intraocular lenses (IOL), while a preferred device is contact lenses, especially the soft contact lenses and scleral lenses with little movement while blinking. The present method and devices can also be applied for orthokeratology to reshape the cornea temporarily, or for setting the parameters in refractive surgery to reshape the cornea permanently for correction of the refractive errors and peripheral ocular defocus together. Spectacles do not move with the eyeballs and it is hard to aim the eyes' center to the center zone. If the ADF device is a spectacle lens, there should be a 2-4 mm single-vision center optical zone 20 for the pupil center, and an ADF zone 21 having a spherical curve with a focus forward or backward from the center optical zone 20. Alternatively, an aspheric curve with the predetermined ±e-value progressively forward or backward in focus from the central zone 20 can be used. The ADF zone 21 is an annular zone radially outward from the outer margin of the center optical zone 20. The vertical meridian of the ADF zone 21 can be set less aspheric (lower plus or less minus e-value respectively) than that of the horizontal meridian for better vision quality. It is preferred to make the ADF zone 21 a horizontal aspheric band, while leaving the vertical meridian single-vision with zero e-value to satisfy the horizontal Hering-Hillebrand deviation for fusion. The two perpendicular meridians can be blended with progressively changing e-values to form a smooth optical surface.

An ADF contact lens can rotate on the eyes, so the zones of the lens are preferably rotationally symmetrical, and the ADF zone 21 of a contact lens can be a single vision annular zone having a spherical curve with a focus more forward or backward than the center zone, while it is preferred to have an aspheric annular zone with the predetermined ±e-value progressively forward or backward in focus from the central zone 20. The aspheric annular ADF zone 21 can be radially outward from the outer margin of the 0.5-1.5 mm single-vision center zone, or alternatively, the two zones can be merged with an e-value for a continuous curve to prevent image jump at the junction. The vertical meridian of the ADF zone 21 of a contact lens 10 can be set less aspheric (lower plus or less minus e-value respectively) than that of the horizontal meridian for better vision quality. It is preferred to make the ADF zone 21 a horizontal aspheric band, while leaving the vertical meridian single-vision with zero e-value to satisfy the horizontal Hering-Hillebrand deviation for fusion. The two perpendicular meridians (0 degree and 90 degrees) can be blended with progressively changing e-values to form a smooth optical surface. A stabilization structure, (prism ballast, truncation, or dynamic stabilization) will be required for aligning the anti-defocus band to the right axis, which is a well know technology for the rigid and soft contact lens manufacturer in producing the toric rigid or soft contact lenses 10.

### Myopia Control

Traditional methods for myopia management or control usually suggested relieving accommodation with cycloplegics, such as atropine, or by using bifocal spectacles with ADD powers for near tasks. The peripheral ADF devices in the present invention can be very useful in slowing the progression of near sightedness, i.e., myopia management. In the present invention, the peripheral anti-defocus is proposed to be a factor to remediate the visual efficiency problems by correcting the image shell mismatch and facilitate binocular fusion. Myopia may progress rather rapidly in cases with visual efficiency problems undiscovered or not remediated. The hyperopic ocular defocus at periphery may induce convergence insufficiency (CI) and a myopic ocular defocus at periphery may induce convergence excess (CE), while both conditions may induce myopia. If the visual efficiency evaluation discovers a CI condition, a hyperopic anti-defocus (H-ADF) device may be applied to slow down or halt the myopia progression. If the visual efficiency evaluation discovers a CE condition, a myopic anti-defocus (M-ADF) device may be applied to slow down or halt the myopia progression instead. A M-ADF lens of the present invention is similar to but different from the center near (CN) multifocal lens. The regular CN multifocal lens has a central zone for near vision with a plus add power, and a peripheral far zone adjacent to and radially outward from the center zone for far vision, which is less plus or more minus in power than the center zone. The M-ADF lens in the present invention also has a center optical zone 20 but the power is for far vision without near add power, while the outer M-ADF zone 21 is made even less plus or more minus in power than the center optical zone 20 for anti-defocus and remediation of the myopic peripheral ocular defocus.

Both CI and CE conditions may induce myopia progression with different mechanisms. In the present invention, myopia is regarded as being caused by strenuous binocular fusion, not the guidance of peripheral defocus towards the ideal optical state as in the prior art. The present invention explains well the research showing that the myopia cases with esophoria in near distance (CE) can be improved for less myopia progression with the peripheral backward multifocal contact lenses that will induce more peripheral hyperopic ocular defocus, which is contrary to what was taught in US Patent Publication No. 20070115431A1, for example.

Hence, binocular efficiency status is checked for myopia cases in the present method, and is tested with the H-ADF or M-ADF testing sets, to determine the ocular defocus at periphery and the ADP strength for remediation as aforementioned. Practitioners may also prescribe an ADF device in empirical ADP for initial adaptation at home and finetune the lens design or start VT in 1-2 months for the residual abnormality. The empirical ADP of an ADF zone 21 is usually 1 to 1.5 diopters per prism to be compensated classically. For example, if a CI case requires a relieving prism of 10 ΔBI (base in) at 40 mm, the ADP of an empirical H-ADF contact lens 10 would be +10 to +15 diopters at the outermost margin of the progressive ADF zone 21. A pair of empirical ADF contact lens 10 can be dispensed for use at home and return to clinic for reevaluation of the visual efficiency in 1-2 months, and fine tune the lens design, ADP or add VT for further improvement of residual dysfunction. Then the optical device, either a pair of spectacles or contact lenses, will be given to remediate the mismatch of the central and peripheral ocular image shells for precise binocular fusion and myopia management.

### Anti-Defocus Spectacle Lens

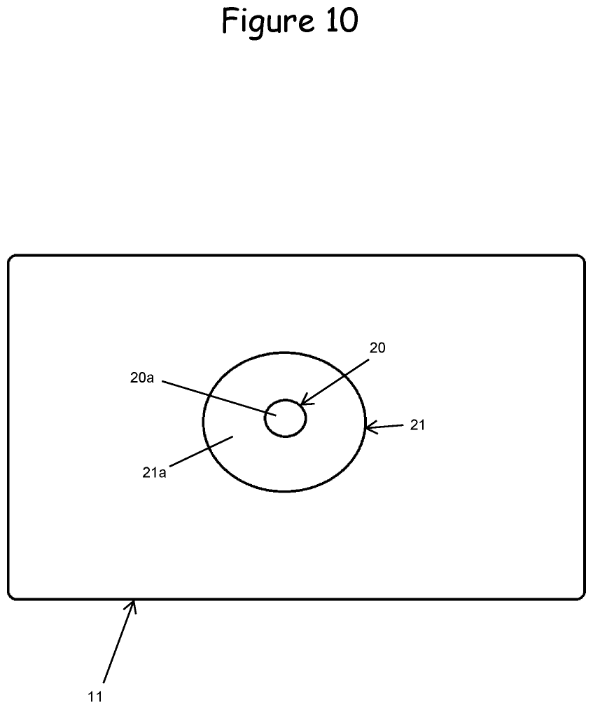

Spectacle lenses according to the present invention (as illustrated in FIG. 10) have a convex front surface with a spherical curved center optical zone 20, and an ADF zone 21. The concave back surface of the ADF spectacle lens is made spherical lens, aspheric or toric to work with the front center optical zone 20 for correction of refractive error, as is well known to spectacle lens manufacturers. The ADF zone 21 can alternatively also be designed on the back surface of the spectacle lens.

In a H-ADF spectacle lens, the center optical zone 20 is preferred to be a spherical power created on the front (convex) surface, with a diameter of 1.5-4.0 mm and an annular more plus H-ADF zone 21 of 8-12 mm to each side of the center optical zone 20 with a total diameter of 18-28 mm, for using at a vertex distance of 12-14 mm. The ADP power difference between the center optical zone 20 and the outermost portion of the H-ADF zone 21 is between +1.00 D and +20 D. The H-ADF zone 21 can be made progressively plus in power radially outwardly with a minus e-value (p-value>1) on the convex front surface for zone 21; or with a plus e-value (p-value<1) on the concave back surface. The foci of the H-ADF zone 21 are progressively shorter along the horizontal meridian, to the shortest (least minus or most plus in power) focal length at its outermost margin, while the vertical meridian is formed zero e-value for single-vision curvature with a constant focal length. The horizontal and vertical meridians are merged with progressively changing e-value for a smooth aspheric surface. The strength of the anti-defocus power (ADP) can be controlled with gradient e-values between ±0.1c-±2.0 e for a forward (myopic) peripheral focusing relative to the baseline without the H-ADF lens. The substantial ADP effect of the spectacle lens measured on eye 14 is minimum-0.50 diopters more myopic at 10 degrees to each side of the fovea retina (N10 and T10), and progressively increases up to minimum-2.00 diopters at 20 degrees to each side of the fovea retina (N20 and T20).

In a M-ADF spectacle lens, the center optical zone 20 is preferred to be a spherical power created on the front (convex) surface, with a diameter of 1.5-4.0 mm and an annular more minus M-ADF zone 21 of 8-12 mm to each side of the center optical zone 20 with a total diameter of 18-28 mm, for using at a vertex distance of 12-14 mm. The ADP power difference between the center optical zone 20 and the outermost portion of the M-ADF zone 21 is between −1.00 D and −20 D. The M-ADF zone 21 can be made progressively minus in power radially outwardly with a plus e-value (p-value<1) on the convex front surface, or with a minus e-value (p-value>1) on the concave back surface. The foci of the M-ADF zone 21 are progressively longer along the horizontal meridian, to the longest (most minus or least plus in power) focal length at its outermost margin, while the vertical meridian is formed zero e-value for single-vision curvature with a constant focal length. The horizontal and vertical meridians are merged with progressively changing e-value for a smooth aspheric surface. The strength of the anti-defocus power ADP can be controlled with gradient e-values between ±0.1 e-±2.0 e. The substantial ADP effect of the spectacle lens measured on eye 14 is minimum +0.50 diopters more hyperopic at 10 degrees to each side of the fovea retina (N10 and T10), and progressively increases up to minimum +2.00 diopters at 20 degrees to each side of the fovea retina (N20 and T20).

### Anti-Defocus Contact Lenses

FIGS. 3-5 illustrate a contact lens 10 designed according to the present invention that can be soft, rigid, corneal, or scleral contact lenses formed from standard contact lens materials. As shown in FIGS. 1-2, the contact lens 10 is adapted to be worn over the cornea 12 of a patient's eye 14. As shown in FIG. 3, the contact lens 10 has at least three correction zones on the convex front surface of the contact lens 10, listed from the center of the lens 10 to the outer periphery: a center optical zone (for far vision) 20, an ADF zone (to adjust ocular defocus) 21, and an intermediate zone 24 which can be a lenticular zone. The concave back surface of such an anti-defocus (ADF) contact lens 10 can be a spherical, aspheric, dual geometry, or reverse geometry lens design.

The optical zone 20 has a back surface that is defined by the base curve 30b, and a front surface that is defined by the center curve 30a and ADF curve 31a. The front optical zone 20 in the present invention is divided into at least two concentric zones. The optical zone 20 is a center zone with a center curve 30a on the front surface and is designed with a refractive power for correcting distant vision. Located outwardly from the optical zone 20 is the ADF zone 21 with an ADF curve 31a on the front surface which is designed with a refractive power for correcting myopia or hyperopic peripheral ocular defocus. The difference between the center optical zone 20 and ADF zone 21 is the anti-defocus power (ADP) for remediation of the ocular defocus.

Although it is possible to create two adjacent annular zones for the center optical zone 20 and ADF zone 21 respectively, distinct small zones with a significant difference in powers may induce image jump, confusion or double vision. In a preferred embodiment, the two zones 20a and 21a are therefore merged with a continuous aspheric curvature with a plus or a minus eccentricity value for a smoother transition.

The optical zone 20 and ADF zone 21 are together preferably about 3-8 mm in diameter, more preferably 6 mm, i.e., 3 mm to each side of the geometric center of the lens. The lens preferably has a progressively steeper or flatter aspheric front optical curve 30a and ADF curve 31a, radially outward from the geometric center of contact lens 10. The maximum anti-defocus power (ADP) for the outermost (most peripheral) margin of the ADF zone 21 is preferably from +3 diopters to +30 diopters in a H-ADF lens; or from −3 diopters to −30 diopters in a M-ADF lens, for different conditions of image shell mismatch. The front center optical zone 20a and front ADF zone 21a can be merged smoothly, for a continuous front center-ADF zone 20a-21a with an aspheric front center-ADF curve 30a-31a to ensure a clear central far image forming at the fovea area of the eye 14, and remediation of the image mismatch at the parafovea, perifovea and peripheral portion of the retina. The formula to calculate the e-value for merging the two zones is e=SIGN(R_{A}−R_{B})*SQRT((R_{A}²−R_{B}²))/(Zone A+Zone B), where R_{A} is the radius of curvature for center optical zone and R_{B} is the radius of curvature for ADF zone 21a. (Zone A+Zone B) is the half zone width of each of the two zones, i.e., the center optical zone 20 and ADF zone 21.

The e-value for merging the two zones 20a and 21a for anti-defocus with ADP +3 to +30 D for the front surface is usually −0.7 e to −3.0 e and that of ADP −3 to −30 D for the front surface is usually +0.7 to +3.0 e, using contact lens materials having refraction index of about 1.4 to 1.6. The contact lens 10 has to be precisely centered for the fovea to perceive light rays from the center optical zone 20a for less spherical aberration.

For a hyperopic anti-defocus contact lens 10, the front surface of the lens can have a center optical zone 20 with a diameter of 0.5-1.0 mm and an annular more plus ADF zone 21 (in this case an H-ADF zone) which is 3-4 mm to each side of the center optical zone 20 for a total diameter of 6-10 mm (i.e., the diameter of the optical zone 20 and ADF zone 21 together). The ADP power difference between the center optical zone 20 and the outermost portion of the H-ADF zone 21 is between +1.00 D and +30 D. The H-ADF zone 21, adjacent to and starting with the same power of the center optical zone 20 in horizontal meridian 51, is made progressively more plus or less minus in power along the horizontal meridian 51 with a negative e-value (p-value>1). The foci of the H-ADF zone 21 are progressively shorter along the horizontal meridian, to the shortest focal length (least minus or most plus in power) in its outermost margin (periphery 21c), while the vertical meridian 52 is formed with a zero e-value (p-value=1) for single-vision curvature. The center optical zone 20 and ADF zone 21 can also be merged with certain e-value to become a continuous center-ADF zone 20-21 as aforementioned. The horizontal and vertical meridians 50 of the center-ADF zone 20-21 are merged with progressively changing e-value for a continuous smooth aspheric surface. The strength of the anti-defocus power ADP can be controlled with gradient e-values of between ±0.1 e-±3.0 e. The substantial ADP effect of the contact lens 10 measured on an eye 14 is a minimum of −0.50 diopters more myopic at 10 degrees to each side of the fovea retina (N10 and T10), and progressively increases up to a minimum of −2.00 diopters at 20 degrees to each side of the fovea retina (N20 and T20).

For a myopic anti-defocus (M-ADF) contact lens 10, the front surface of the M-ADF lens 10 can have a center optical zone 20 with a diameter of 0.5-1.0 mm and an annular, more minus ADF zone 21 (in this case an M-ADF zone) which is 3-4 mm to each side of the center optical zone 20 for a total diameter of 6-10 mm (i.e., the diameter of the optical zone 20 and the ADF zone 21 together). The ADP power difference between the center optical zone 20 and the outermost portion of the M-ADF zone 21 is between −1.00 D and −30 D. The M-ADF zone 21, adjacent to and starting with the same power of the center optical zone 20 in the horizontal meridian 51, is made progressively more minus or less plus in power along the horizontal meridian 51 with a positive e-value (p-value<1). The foci of the M-ADF zone 21 are progressively longer along the horizontal meridian, to the longest focal length (least plus or most minus in power) in its outermost margin (periphery 21c), while the vertical meridian 52 is formed with a zero e-value (p-value=1) for single-vision curvature. The center optical zone 20 and ADF zone 21 can also be merged with a certain e-value as aforementioned to become a continuous center-ADF zone 20-21. The horizontal and vertical meridians 50 of the center-ADF zone 20-21 are merged with a progressively changing e-value for a continuous smooth aspheric surface. The strength of the anti-defocus power ADP can be controlled with gradient e-values between ±0.1 e-±3.0 e. The substantial ADP effect of the contact lens 10 measured on eye 14 is minimum +0.50 diopters more hyperopic at 10 degrees to each side of the fovea retina (N10 and T10), and progressively increases up to a minimum of +2.00 diopters at 20 degrees to each side of the fovea retina (N20 and T20).

Referring to FIGS. 3-5, myopic contact lenses 10 tend to get thicker toward the peripheral edges with higher powers. To reduce the peripheral edge thickness of the present ADF contact lenses 10, a lenticular curve steeper than the curves 30a or 31a of zones 20 and 21 can be incorporated radially outwardly from the ADF zone 21. Contrary to myopic lenses, emmetropic, low myopic or hyperopic contact lenses 10 may get too thin at their edges and thus crack or chip more easily than is desirable, so to increase the peripheral edge thickness of the ADF contact lenses 10, a lenticular curve that is flatter than the curves 30a or 31a can be incorporated radially outwardly from the ADF zone 21a.

One or more optional intermediate zones within zone 24 with a half zone width of 2.0-5.0 mm radially outward can also be added between the front ADF zone 21a and a lenticular curve for optical or therapeutic reasons. For example, the intermediate zone 24 can be added for the same correcting power of the center optical zone 20 that can further enhance the peripheral far incoming light for better distance vision in the nighttime.

The different radii used to define the base curve in the contact lens 10, i.e. the base curves of the optical zone 20, ADF zone 21, connecting zone 26, and peripheral zone 28 and their relative thicknesses are calculated after careful examination of a patient's eye and the associated ocular tissue. The corneal curvature must be measured, the proper contact lens power defined, and the anticipated physiological response to the contact lenses 10 must be determined. An individual skilled in the examination techniques of the ocular system is capable of performing these tasks.

### Anti-Defocus Orthokeratology

It is also an object of the present invention to provide an orthokeratology contact lens 10 that provides effective remediation of peripheral ocular defocus. It is another object of the present invention to provide an orthokeratology contact lens 10 to correct refractive errors, including and not limited to hyperopia, myopia, presbyopia and astigmatism, which is required for correction of a visual efficiency problem.

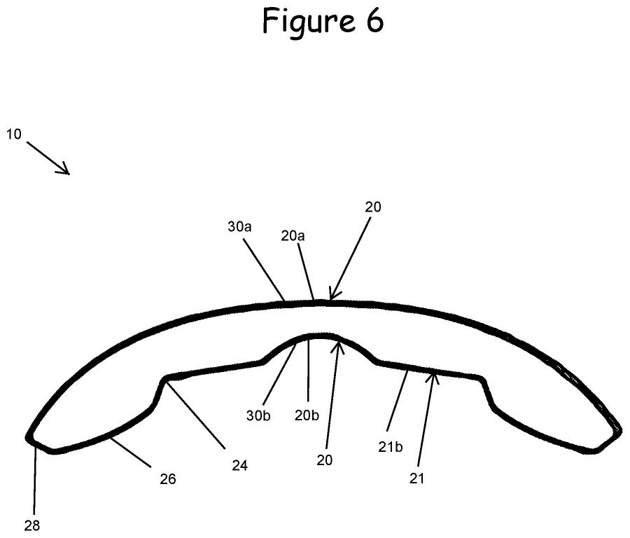

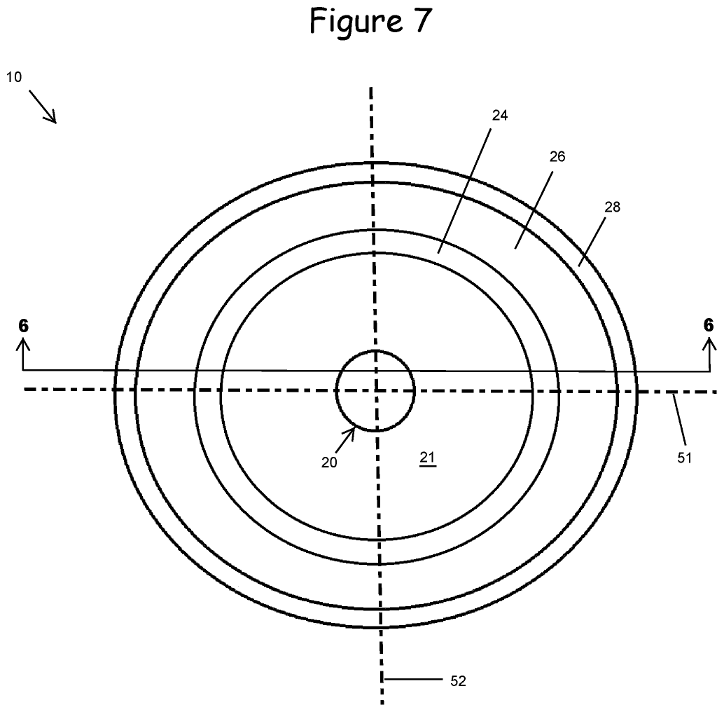

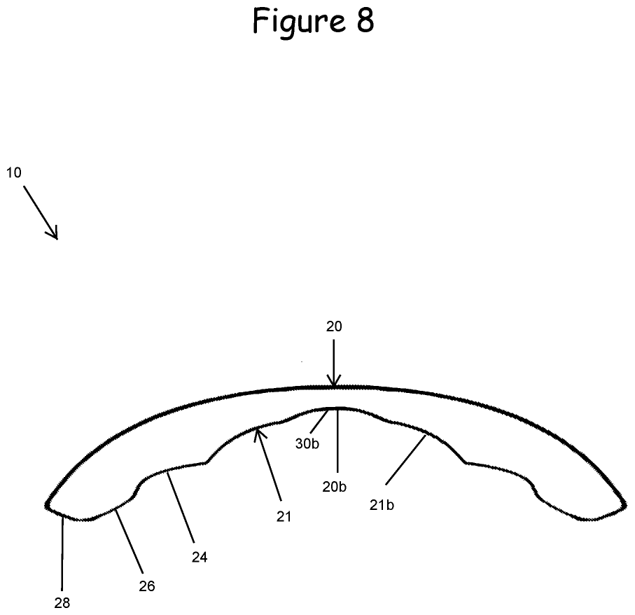

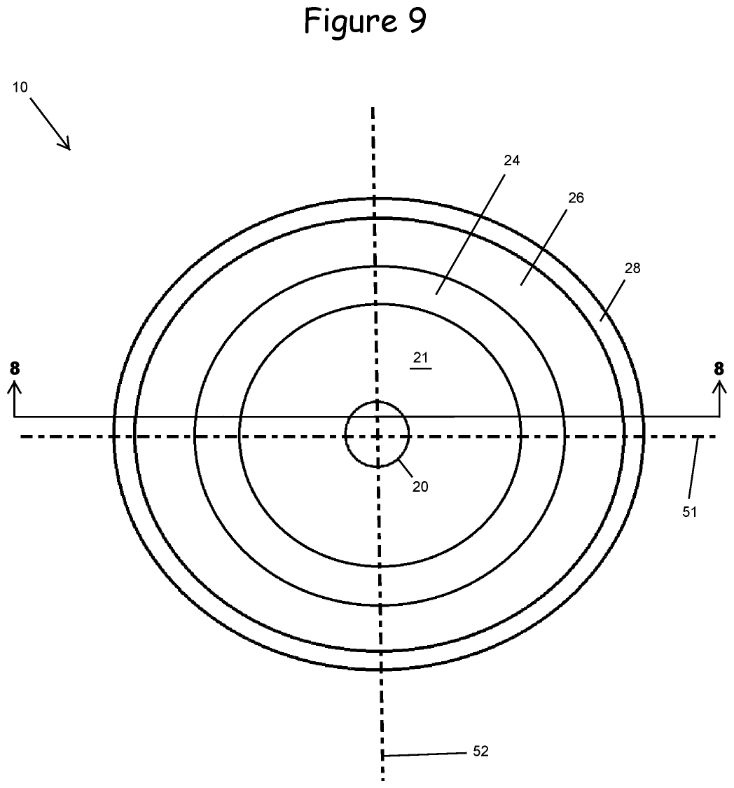

These objects of the present invention can be achieved by providing an apparatus and method for correcting the refractive error with a peripheral ocular defocus condition in a patient's eye. In accordance with a method of the present invention, an anti-defocus (ADF) orthokeratology contact lens 10 (as shown for example in FIGS. 6-9) is fitted to a cornea of a patient's eye, the contact lens 10 having a back surface with plurality of zones that includes a central optical zone having front and back surfaces (20a, 20b) with corresponding curves (30a, 30b) respectively; an ADF zone 21 with an ADF base curve 31b, an intermediate zone 24 (plateau zone in hyperopic lenses), a connecting or fitting zone 26, and an alignment zone and/or a peripheral zone 28. An ADF base curve 21b is carefully created to flatten or steepen the mid-peripheral cornea curvature to cause the cornea 12 to have an anti-defocus mid-peripheral portion surrounding the central zone created by the base curve 20b of the optical zone 20 for far vision. The target power of the optical zone 20 for correction of the far vision can be determined by an eye care practitioner for fitting orthokeratology lenses. The shape of the ADF zone (21a, 21b) can be derived from testing the ocular defocus with an ADF testing spectacle lens or an ADF testing contact lens as aforementioned.

In accordance with an apparatus of the present invention, a contact lens 10 is provided having a optical zone curve (30a, 30b) portion of the lens, an ADF curve (31a, 31b) portion of the lens circumscribing and coupled to the optical zone curve portion, a plateau curve in an intermediate zone 24 and/or a fitting curve in a connecting zone 26 portion of the lens circumscribing and coupled to the ADF curve (31a, 31b) portion, and an alignment curve and/or a peripheral curve in a peripheral zone 28 portion of the lens circumscribing and coupled to the intermediate zone 24 or connecting zone 26 portion.

The diameter of the central optical zone 20 of the contact lens 10 can preferably be varied from 1.0-3.0 mm for different purposes of correcting myopia, hyperopia or presbyopia. The zone width of the ADF zone 21 can preferably be varied from 1.0 to 4.0 mm for reshaping the predetermined myopic or hyperopic peripheral ocular defocus on a corneal surface. The total diameter of the optical zone 20 and ADF zone 21 together is preferred to be 4.0-8.0 mm, which can be merged for an aspheric optical-ADF curve with an eccentricity value of −0.1 e to −3.0 e for a H-ADF orthokeratology contact lens 10 to mold off hyperopic ocular defocus, or +0.1 e to +3.0 e for a M-ADF orthokeratology lens 10 to mold off myopic ocular defocus on the cornea 12 of the eye 14.

An anti-defocus (ADF) orthokeratology contact lens 10 can be a spherical, aspheric, dual geometry, and/or reverse geometry design as taught in U.S. Pat. Nos. 6,652,095; 7,070,275; and 6,543,897 for orthokeratology RGP (rigid gas permeable) lenses.

For treating the myopic person with peripheral hyperopic ocular defocus, the base curve 30b should preferably be flatterer than the central cornea curvature. The center optical zone 20 and ADF zone 21 should be wider and preferably 3-4 mm for better far vision. For treating the hyperopic person, the base curve 30b should preferably be steeper than the central cornea curvature. For treating the presbyopic person, the center optical zone 20 can be divided into two portions. An inner optical zone should be designed to be very small for the purpose of near vision to prevent it from hindering far vision, while an outer optical zone then should be slightly wider to mold the juxta-central cornea area into a flatter zone to clear up far vision (reducing myopia, hyperopia, or astigmatism if any).

The different radii used to define the base curves (30a, 30b) in contact lens 10 and the ADF curves (31a, 31b), as well as the curves for the intermediate zone 24, the connecting zone 26, and the peripheral zone 28 and their relative thicknesses, are calculated after careful examination of a patient's eye 14 and the associated ocular tissue. The corneal curvature must be measured, the proper contact lens power defined, and the anticipated physiological response to the contact lenses 10 must be determined. An individual skilled in the examination techniques of the ocular system is capable of performing these tasks.

### Anti-Defocus Refractive Surgery

It is another object of the present invention to provide a method for designing the parameters in performing refractive surgery (LASIK/LASEK) on the cornea 12. The anti-defocus parameters are added for also remediating the peripheral ocular defocus to improve visual efficiency problems, while correcting the hyperopia, myopia, presbyopia and astigmatism by surgery. Similar to performing orthokeratology, the shape of the ADF zone 21 can be derived from testing the ocular defocus with an ADF testing spectacle lens or an ADF testing contact lens as aforementioned, before performing surgery. The corneal shape so designed forms a treatment zone having a central zone preferably varying from 1.0-3.0 mm for different purposes of correcting myopia, hyperopia or presbyopia. The zone width of the ADF zone 21 adjacent to and radially outward from the central zone 20 can be varied from 1.0 to 4.0 mm for correcting the predetermined myopic or hyperopic peripheral ocular defocus on the corneal surface. The total diameter of the center optical zone 20 and ADF zone 21 is preferably 5.0-8.0 mm, which can be merged for an aspheric center-ADF zone 20-21 with an eccentricity value of −0.1 e to −3.0 e for correcting hyperopic ocular defocus, or ±0.1 e to ±3.0 e for correcting myopic ocular defocus on the cornea 12 of the eye 14.

The LASIK/LASEK surgery can be a complementary surgery for the eyes post-cataract surgery with mismatch of the central and peripheral images for inserting different type of IOLs in fellow eyes. The strength of the ocular defocus can be determined using an ADF testing set as aforementioned and performing LASIK/LASEK surgery for remediation.

### EXAMPLES

### Example 1

Case 1 is an 11 year old female who had exotropia (XT) with one eye shifted outward since 2 y/o. She received strabismus surgery at 7 y/o and unfortunately developed esotropia (ET), with the right eye squinting inward, manifesting diplopia and developing myopia shortly after surgery. The manifestation of diplopia after developing ET is caused by disruption of the sensory adaptation formed for XT with suppression scotoma. We prescribed 55% methafilcon soft H-ADF contact lenses and surprisingly found the eye position straightened in both near and far distances with no more diplopia. The myopia also stopped progressing for 4 years since wearing the anti-defocus contact lenses.

### <Right Eye ADF Soft Contact Lens>

Center power: −1.25 D (Myopia)
Center-ADF zone 20-21: BOZ (zone width) 8.5 mm,
   BOZR (radius of curvature) 9.0 mm
   FOZR (Center front curve): 9.54 mm
   Front ADP: +10 D
   Horizontal front e-value −1.11 (p=2.23)
Intermediate zone 24-26: half zone width 1.16 mm, radius of curvature 7.28 mm
Peripheral zone 28: half zone width 1.0 mm, radius of curvature 9.8 mm

### <Left Eye>

Center power: −0.75 D (Myopia)
Center-ADF zone 20-21: BOZ (zone width) 8.5 mm,
   BOZR (radius of curvature) 9.0 mm
   FOZR (Center front curve): 9.43 mm
   Front ADP: +10 D
   Horizontal front e-value −1.08 (p=2.17)
Intermediate zone 24-26: half zone width 1.16 mm, radius of curvature 7.28 mm
Peripheral zone 28: half zone width 1.0 mm, radius of curvature 9.8 mm

Following the peripheral ocular defocus theory, the ET was surgically induced while the basic problem is still XT. The XT suppression scotoma for sensory adaptation was disrupted by surgery, turning into ET with manifest diplopia and developing myopia. It is assumed the inborn hyperopic ocular defocus induced XT while the surgery adjusted the extra ocular muscle to turn the eyes inward without correcting the hyperopic ocular defocus. The peripheral image shell does not satisfy Panum's area for fusion that is required for straightening the eyes and turning them inward after surgery, which in turn, disrupted the suppression scotoma formed for XT sensory adaptation with manifestation of diplopia and myopia progression. While the H-ADF contact lenses corrected the inborn hyperopic ocular defocus for better binocular fusion and straightened the eyes, without regard to the eye position being XT or ET. This case strongly indicated that the strabismus of this case is secondary to peripheral ocular defocus, while myopia is tertiary to the strenuous efforts for fusion with the induced diplopia after surgery, which explains how H-ADF contact lenses improved eye teaming and stopped myopia progression simultaneously.

### Example 2

Case two is a 9 y/o boy diagnosed as having ADD (attention deficit disorder) and taking Concerta everyday, though it did little to help with reading comprehension. He had a birth history of meconium aspiration with resuscitation, but this was uneventful until school age and when it was discovered that he was unable to study. He was found to have severe eye tracking problems by an optometrist and was referred for visual efficiency evaluation. The SCCO test revealed rather normal “fixation maintenance” but total failure in Pursuit & Saccade. His refraction was emmetropia in the right eye and mild +0.50 D hyperopia in the left eye. The OEP 21-point examination revealed convergence insufficiency (CI). While doing the SCCO test, he failed to follow the target, with constant upshifting of both eyeballs while checking pursuit and overflow phenomenon, with both hands tensely stretched. With the severe oculomotor dysfunction and CI that is very unusual for VT, we decided to try H-ADF soft contact lenses and follow up in 1-2 months before doing visual training (VT).

### <Right Eye H-ADF Soft Contact Lens>

Center power: plano (zero power)
Center-ADF zone 20-21: BOZ (zone width) 8.5 mm,
   BOZR (radius of curvature) 9.0 mm
   FOZR (Center front curve): 8.92 mm
   Front ADP: +10 D
   Horizontal front e-value −1.06 (p=2.12)
Intermediate zone 24-26: half zone width 1.16 mm, radius of curvature 7.28 mm
Peripheral zone 28: half zone width 1.0 mm, radius of curvature 9.8 mm

### <Left Eye H-ADF Soft Contact Lens>

Center power: +0.50 (Myopia)
Center-ADF zone 20-21: BOZ (zone width) 8.5 mm,
   BOZR (radius of curvature) 9.0 mm
   FOZR (Center front curve): 9.17 mm
   Front ADP: +10 D with
   Horizontal front e-value −1.05 (p=2.10)
Intermediate zone 24-26: half zone width 1.16 mm, radius of curvature 7.28 mm
Peripheral zone 28: half zone width 1.0 mm, radius of curvature 9.8 mm

We rechecked the SCCO Pursuit & Saccade after wearing the H-ADF contact lenses for 2 months. His face appeared calm and relaxed throughout the testing. There was no more overflow phenomenon of the extremities during the test. He could follow the moving target very well except for losing places occasionally, with no more eyeball upshifting. This showed that peripheral ocular defocus can induce binocular misalignment as well as oculomotor dysfunction that is usually misdiagnosed as a neurological or psychological problem and treated inappropriately. With significant improvement in oculomotor function, we arranged VT to further fine tune the visual skills.

### Example 3

Case 3 is a 25 y/o male with reading problems and excessive myopia. He had been in front of his class until 17 y/o in the 11^{th} grade, when it became difficult for him to read due to severe headaches, with words waving after reading for 10 minutes. Myopia also progressed extremely quickly with studying harder in school. He tried atropine, RGP and reading spectacles to stop myopia progression but in vain. He also tried VT but it did little to help. The condition induced anxiety and insomnia and was diagnosed as depression with a need for an antidepressant.

### <Initial OEP 21-Point Examination>

Refraction (#7): Right eye −15.25 −3.00×170° (Myopia −15.25 D with astigmatism 3.0 D)
   Left eye −15.00-0.75 @ 0° (Myopia −15.00 D with astigmatism 0.75 D)
Phoria at Far (#8) 10Δ XP (exophoria)
Phoria at Near (#13) 25Δ XP (exophoria)
Fixation Disparity (Wesson card): OD suppression at near
Accommodation power: OD 4.00 D, OS 3.50 D
(Other visual efficiency data are unavailable due to profound OD suppression)

The diagnosis was convergence insufficiency (CI) and accommodation insufficiency (AI) that had never been diagnosed and managed, which could be also the reason for rapid myopia progression.

He was living out of town and could not come for traditional VT. We decided to fit H-ADF soft contact lenses to improve the accommodation insufficiency, convergence insufficiency and hopefully to slow down myopia progression.

### <Right Eye H-ADF Soft Contact Lens>

Center power: −13.50 (myopia 13.50 diopters)
Center-ADF zone 20-21: BOZ (zone width) 8.5 mm,
   BOZR (radius of curvature) 9.0 mm
   FOZR (Center front curve): 13.43 mm
   Front ADP: +25 D
   Horizontal front e-value −2.18 (p=5.84)
Intermediate zone 24-26: half zone width 1.16 mm, radius of curvature 7.28 mm
Peripheral zone 28: half zone width 1.0 mm, radius of curvature 9.8 mm

### <Left Eye Contact Lens>

Center power: −13.50 D (myopia 13.50 diopters)
Center-ADF zone 20-21: BOZ (zone width) 8.5 mm,
   BOZR (radius of curvature) 9.0 mm
   FOZR (Center front curve): 13.43 mm
   Front ADP: +25 D
   Horizontal front e-value −2.18 (p=5.84)
Intermediate zone 24-26: half zone width 1.16 mm, radius of curvature 7.28 mm
Peripheral zone 28: half zone width 1.0 mm, radius of curvature 9.8 mm

The pair of ADF lenses reduced the near exophoria dramatically and instantly. The far 10ΔXP reduced to 6ΔXP and near 25ΔXP was reduced to 12ΔXP. The fixation disparity was checked immediately with the pair of ADF lenses, revealing no more OD ICS with 9Δassociated XP (exophoria) at 40 cm.

We let him go home with H-ADF soft contact lenses for daytime wear and did not do VT. He reported that he could wear the contact lenses and study comfortably without other aid for whole day with no headache. We rechecked his visual efficiency 11 months after wearing the H-ADF lenses and found no myopia progression and nearly normal visual efficiency data.

<Recheck OEP 21-Point Examination (11 Months after Initial Exam.)>
Refraction (#7): Right eye −15.25 −3.00×170° (Myopia −15.25 D with astigmatism 3.0 D)
   Left eye −15.00 −0.75 @ 0° (Myopia −15.00 D with astigmatism 0.75 D)

### <Visual Efficiency Checked Wearing the H-ADF Soft Contact Lenses)

Phoria at Far (#8) 5Δ XP (exophoria)
Phoria at Near (#13) 12Δ XP (exophoria)
Vergence at near: #16A 12Δ; #16B 18/10Δ
   #17A 12Δ; #17B 24/15Δ
Fixation Disparity (Wesson card): zero disparity
Accommodation power: OD 4.25 D, OS 4.25 D

This case shows that peripheral hyperopic ocular defocus is the etiology of convergence insufficiency. The ADF device can amend the optical abnormality and improve CI. Long term usage of the ADF device can improve the motor range (#16 and #17) as well as binocular fusion, which in turn can cure dyslexia and stop myopia progression.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

1. A lens for correcting peripheral ocular defocus, the lens having a front surface and a back surface, comprising:
a center zone in a central portion of the lens having a lens power for correcting refractive errors; and
an aspheric, annular anti-defocus (ADF) zone adjacent to and extending radially outwardly from the center zone,
wherein the lens is a spectacle lens or a contact lens, and
wherein the front surface or the back surface of the lens has a horizontal meridian and a vertical meridian, the horizontal meridian and the vertical meridian each having an e-value, wherein the vertical meridian of the ADF zone is less aspheric than the horizontal meridian of the ADF zone, and wherein the curvature of the surface of the lens between the horizontal meridian and the vertical meridian is blended with progressively changing e-values to form a smooth optical surface.

2. The lens of claim 1, wherein the vertical meridian of the ADF zone has a zero e-value.

3. The lens of claim 1, wherein the e-value of the vertical meridian is less than ½ of the e-value of the horizontal meridian.

4. The lens of claim 1, wherein the vertical meridian of the ADF zone is a single-vision curve having the same power as the central zone.

5. The lens of claim 1, wherein the lens is a spectacle lens, wherein the horizontal meridian and the vertical meridian are on the front surface of the lens, wherein the center zone has a diameter of between 1.5 and 4.0 mm, and wherein the center zone and ADF zone together have a diameter of between 18 and 28 mm.

6. The spectacle lens of claim 5, wherein the horizontal meridian of the ADF zone is aspheric and progressively plus in power radially outward from an inner boundary of the ADF zone and has an anti-defocus power (ADP) of +1.00 to +20.0 diopters, ADP being defined as a power difference between an outer periphery of the ADF zone and an outer periphery of the center zone, wherein the lens is useful in the treatment of hyperopic ocular defocus.

7. The spectacle lens of claim 5, wherein the horizontal meridian of the ADF zone is aspheric and progressively minus in power radially outward from an inner boundary of the ADF zone and has an anti-defocus power (ADP) of −1.00 to −20.0 diopters, ADP being defined as power difference between an outer periphery of the ADF zone and an outer periphery of the center zone, wherein the lens is useful in the treatment of myopic ocular defocus.

8. The lens of claim 1, wherein the lens is a contact lens, wherein the center zone has a diameter of between 0.5 and 1.0 mm, wherein the ADF zone extends radially outwardly from the center zone for at least 3 to 4 mm, and wherein the center zone and annular ADF zone together have a diameter of between 6 and 10 mm.

9. The contact lens of claim 8, wherein the front surface or the back surface of the center zone and the ADF zone each have an e-value, wherein the e-values of the center zone and the ADF zone are merged to form an aspheric center-ADF zone, and wherein the horizontal meridian and the vertical meridian of the center-ADF zone are merged with a rotationally progressive e-value to form a center-ADF zone with a continuous smooth aspheric surface.

10. The contact lens of claim 9, wherein the rotationally progressive e-value Eₓ along an axis X^{o} of the center-ADF zone is derived with the following formula:
$${E}_{x} = SIGN ( {XR}_{p} - {R}_{c} ) * {( ABS ( {XR}_{p}^{2} - {R}_{c}^{2} ) )}^{1 / 2} / d , \qquad ( Equation 2.2 )$$
where XRₚ is the radius of curvature at a point radially outward along axis X^{o} for a distance d, and wherein XRₚ is derived from the following formula:
$${XR}_{p} = {HR}_{p} + {sin ( {X}^{o} )}^{2} * ( {VR}_{p} - {HR}_{p} ) , \qquad ( Equation 2.1 )$$
and where:
R_{c} is the radius of curvature at the center of the contact lens;
HRₚ is the radius of curvature at a point radially outward along the horizontal meridian for a distance d; and
VRₚ is the radius of curvature at a point radially outward along the vertical meridian for a distance “d”.

11. The contact lens of claim 9, wherein the vertical meridian has an e-value of zero and has a single-vision power throughout the center-ADF zone, and wherein the e-value of the horizontal meridian is not zero and is between ±0.1 e and ±3.0 e.

12. The contact lens of claim 11, wherein:
(i) the contact lens is for use in the treatment of hyperopic ocular defocus, wherein the horizontal meridian is progressively plus in power radially outward from a central portion of the lens with an anti-defocus power (ADP) of +1.00 to +30.0 diopters, ADP being defined as power difference between an outer periphery of the ADF zone and an outer periphery of the center zone, wherein the front surface of the horizontal meridian has an e-value of between −0.1 e and −3.0 e, or wherein the rear surface of the horizontal meridian has an e-value of between +0.1 e and +3.0 e; or
the contact lens is for use in the treatment of myopic ocular defocus, wherein the horizontal meridian is progressively minus in power radially outward from a central portion of the lens with an anti-defocus power (ADP) of −1.00 to −30.0 diopters, ADP being defined as a power difference between an outer periphery of the ADF zone and an outer periphery of the center zone, wherein the front surface of the horizontal meridian has an e-value of between +0.1 e and +3.0 e, or wherein the rear surface of the horizontal meridian has an e-value of between −0.1 e and −3.0 e.

13. (canceled)

14. The contact lens of claim 8 for use in performing orthokeratology, wherein the horizontal meridian and the vertical meridian are on the back surface of the lens in order to achieve corneal molding, and wherein the ADF zone has an e-value of between ±0.1 e and ±3.0 e.

15. The contact lens of claim 8, further comprising:
an intermediate zone 24 coupled to and extending radially outwardly from the ADF zone, with a zone width of 2.0-5.0 mm;
a connecting zone 26 coupled to and extending radially outwardly from the intermediate zone 24 for bearing the contact lens on a cornea; and
a peripheral zone 28 coupled to an outer periphery of the contact lens.

16. (canceled)

17. A method for correcting peripheral ocular defocus in a subject's eye for improvement or remediation of visual efficiency problems, comprising the steps of:
(a) determining an anti-defocus power (ADP) for a lens having a center zone in a central portion of the lens and an anti-defocus (ADF) zone adjacent to and extending radially outwardly from the center zone, wherein the center zone has a central focal point to form a central image at the fovea retina for correcting refractive error, wherein ADP is defined as a power difference between an outer periphery of the ADF zone and an outer periphery of the center zone, and wherein the determined ADP is sufficient to offset the peripheral ocular defocus and to realign peripheral images in the subject's eye in order to improve peripheral fusion and visual efficiency; and
(b) providing the lens to the subject.

18. The method of claim 17, wherein determining the anti-defocus power (ADP) further comprises the steps of:
(i) checking baseline visual efficiency data for the subject;
(ii) selecting ADF testing lenses based on the type of visual efficiency problem experienced by the subject;
(iii) testing raw ocular defocus strength by introducing the ADF testing lenses gradually from lower to higher ADP until an optimum ADP is determined which achieves maximum normalization of the visual efficiency data; and
(iv) providing a pair of ADF spectacles or contact lenses having the optimum ADP to the subject.

19. The method of claim 18, further comprising the steps of: repeating steps (i) to (iv) after the subject has worn the provided spectacles or contact lenses for a predetermined period of time.

20. The method of claim 17, wherein;
(i) the horizontal meridian of the ADF zone is aspheric and progressively plus in power radially outward from an inner boundary of the ADF zone, wherein the ADF zone has an ADP of +1.00 to +20.0 diopters, and wherein the lens is used for remediation of binocular efficiency problems or for myopia control in cases having hyperopic ocular defocus; or
(ii) wherein the horizontal meridian of the ADF zone is aspheric and progressively minus in power radially outward from an inner boundary of the ADF zone, wherein the ADF zone has an ADP of −1.00 to −20.0 diopters, and wherein the lens is used for treatment of myopic ocular defocus.

21. (canceled)

22. The method of claim 17, further comprising the step of inducing an ADP effect in the subject's eye for a minimum of 0.50 diopters relatively forward (more myopic) or backward (more hyperopic) in peripheral foci, measured at 10 degrees to each side (N10 and T10) of the subject's fovea retina or measured at 20 degrees to each side (N20 and T20) of the subject's fovea retina.

23. (canceled)

24. The method of claim 17, wherein a visual efficiency problem selected from the group consisting of oculomotor dysfunction, accommodative dysfunction, vergence dysfunction and abnormal sensory adaptation is treated.
